(19) United States
(12) Patent Application Publication
(10) Pub. No.: US 2025/0222255 A1
(43) Pub. Date: Jul. 10, 2025

(54) SYSTEM, STIMULATION DEVICE AND METHOD FOR CARRYING OUT A STIMULATION PROCESS

(71) Applicant: Drägerwerk AG & Co. KGaA, Lübeck (DE)

(72) Inventors: Marcus EGER, Lübeck (DE); Ronja MÜLLER-BRUHN, Windisch (CH); Harald GENGER, Bad Wimpfen (CH)

(21) Appl. No.: 18/728,921

(22) PCT Filed: Dec. 12, 2022
(86) PCT No.: PCT/DE2022/100939

§ 371 Date: Jul. 15, 2024

(30) Foreign Application Priority Data
Jan. 17, 2022 (DE) 10 2022 100 939.4

(51) Int. Cl. A61N 1/36; A61M 16/00

(52) U.S. Cl. CPC A61N 1/3601; A61M 16/026; A61M 2205/054; A61M 2205/18; A61M 2205/3303; A61M 2205/3334

(57) ABSTRACT

A device and a process for carry out a stimulation of the nervous system for ventilating a living being (33) on the basis of information (45) provided. Data and/or measured values (44) in relation to a state of ventilation of the living being (33) or an operating state of a ventilator (20) are used in order to bring about the manner of stimulation.

### CROSS REFERENCE TO RELATED APPLICATIONS

This application is a United States National Phase Application of International Application PCT/DE2022/100939, filed Dec. 12, 2022, and claims the benefit of priority under 35 U.S.C. § 119 of German Application 10 2022 100 939.4, filed Jan. 17, 2022, the entire contents of which are incorporated herein by reference.

### TECHNICAL FIELD

The present invention pertains to a stimulation system, to a stimulation device and to a process for carrying out a stimulation of the nervous system for ventilating a living being on the basis of information provided, data and/or measured values provided in relation to a state of ventilation of a living being or in relation to an operating state of a ventilator.

### BACKGROUND

In the absence of spontaneous breathing but also in the presence of spontaneous breathing, the electromagnetic or electrical ventilation may take place independently as well as synchronized with the spontaneous breathing. The spontaneous breathing activity of respiratory muscles may be influenced by the stimulation pattern. A stimulation of respiratory muscles for the activation or support of a breathing activity or spontaneous breathing activity offers many different advantages from a medical and clinical point of view. For example, atrophy of the diaphragm can thus be prevented and a need for weaning from a mechanical ventilation can thus often be avoided. In addition, there arises a need for coordination between stimulation and the respiratory feedback. A synchronization of the stimulation with the ventilation leads to advantages during the ventilation. Some examples of advantages of the synchronization should be mentioned here:
avoidance of undesired muscle stimulations during certain phases of exhalation,
support of spontaneous breathing activity with maximum harmony during phases of the breathing cycle with patient-induced breaths, and
avoidance of asynchrony during a ventilation of a patient between a ventilator and the patient during the carrying out or triggering of the ventilation.

Devices for stimulating the nervous system are known from the state of the art. U.S. Pat. No. 11,052,250 thus describes a system and a process for the electrical stimulation of the phrenic nerve, wherein the results of the stimulation with the activation of the diaphragm can be monitored on the basis of the inspiratory work of breathing.

U.S. Pat. No. 5,061,234 shows a magnetic stimulator for stimulating biological tissue. A coil array is operated in resonance with a capacitor, and the stimulation is controlled by means of a control circuit.

WO 2019154839 A1 describes a device for an electromagnetic inductive activation of the nervous system for stimulating muscles. A method for an automatic adaptation of the electromagnetic field with a calibration is described.

US 2019175908 A1 describes a device for activating the nervous system for stimulating muscles by means of an electrode array. A use of additional sensors such as a sensor mechanism and/or a flow rate sensor mechanism is described in connection with the embodiment of the stimulation device or with the carrying out of the stimulation.

### SUMMARY

An object of the present invention is to provide a process for carrying out a stimulation for influencing the nervous system, a stimulation system and a stimulation device for influencing the nervous system.

Another object that is closely linked with this object arises from making possible an improvement during a ventilation of the lungs on the basis of information provided by carrying out the stimulation such that it is adapted to the situation of the living being, to the ventilation of the living being and/or to the operating situation of the ventilator.

These and other objects are accomplished with the features according to the invention.

The object for a process for carrying out a stimulation for influencing the nervous system is accomplished with features disclosed herein.

The object for a computer program or computer program product that may include a computer readable storage medium (or media) having computer readable program instructions thereon for causing a processor to carry out aspects of the present invention. Computer readable storage medium can be a tangible device that can retain and store instructions for use by an instruction execution device for executing the process for carrying out a stimulation for influencing the nervous system.

The object for a device for carrying out the process for carrying out the stimulation for influencing the nervous system is accomplished with features disclosed herein.

The object for a system for a stimulation for influencing the nervous system is accomplished with features disclosed herein.

Advantageous embodiments of the present invention will be explained in more detail in the following description with partial reference to the figures.

Based on information and data provided and/or on measured values provided in relation to a state of ventilation of a living being or of information and data provided and/or of measured values provided in relation to an operating state of a ventilator as at least one piece of information, a stimulation of the nervous system is carried out according to the present invention with an adaptation of the manner of carrying out the stimulation of the nervous system on the basis of the information provided or of the at least one piece of information. The stimulation of the nervous system takes place with action on the phrenic nerve. The stimulation signal is therefore especially suitable and configured for actuating the phrenic nerve, which regulates and/or triggers the breathing or ventilation of the living being. The stimulation signal can be applied to the living being by means of a stimulation device and can be used to produce a spontaneous activity of the living being. The stimulation device may be configured, for example, as an electrode array or as a coil array with arrangement in the head/neck region, the thoracic region (thorax) or the adnominal region (abdomen) with action on the diaphragm. The inclusion of such information, for example and preferably an inclusion of information which can be provided by
a pressure sensor mechanism,
a flow rate sensor mechanism or
a combined pressure/flow rate sensor mechanism, makes possible a measurement-based feedback of the action on the ventilation of the living being.

Information on flow rates, provided, for example, by a flow rate sensor arranged in an inspiratory gas line to the living being, can make possible an adaptation of the stimulation as well as a synchronization between stimulation and breathing or ventilation both for pressure-controlled ventilation modes and for volume-controlled ventilation modes.

The synchronization between stimulation and breathing may in this case be independent from the operating state of the ventilator, especially independent from the fact whether the ventilator is used in a pressure-controlled or volume-controlled mode. In general, it can be described in connection with this that the effect of the stimulation is manifested in the measured variable such that is not influenced by the control of the ventilation by the ventilator.

Synchronization can be achieved by the carrying out of the stimulation according to the present invention between mechanically triggered breaths and breaths triggered spontaneously by the patient, i.e., especially a spontaneous breathing activity with muscle activity triggered by stimulation. An asynchrony between the patient or living being and the ventilator can be avoided in this manner during the breathing/ventilation.

The advantage of avoiding an asynchrony in this manner can be achieved in this case for different variants of ventilation modes, such as pressure-controlled or pressure-regulated and volume-controlled or volume-regulated ventilation modes as well as for different variants of supporting ventilation modes or for ventilation modes with spontaneous breathing support.

The manner of synchronization is based on the ventilation pattern of the ventilator, essentially on whether the ventilator carries out the ventilation on the living being in an operating state with a volume-controlled ventilation mode or in an operating state with a pressure-controlled ventilation mode.

In case of a pressure-controlled ventilation, the ventilation is carried out essentially by feeding breathing gases under an essentially constant, rectangular inspiratory ventilation pressure time course (curve). In case of a volume-controlled ventilation, the ventilation is carried out essentially by feeding breathing gases with a flow rate whose course during the inhalation has essentially a usually constant and rectangular signal shape.

The essential difference between pressure-controlled ventilation and volume-controlled ventilation modes is determined by which of the two parameters, i.e., the ventilation pressure or the volume, is selected as the target variable.

From this arises for the carrying out of the ventilation by the ventilator whether the ventilation pressure and/or a change in the ventilation pressure is controlled, i.e., controlled or regulated during each ventilation cycle or whether the volume or a change in volume over the course of time during each ventilation cycle or the flow rate is controlled, i.e., controlled or regulated, by the ventilator. In other words,
the pattern of the controlled inspiratory flow rate (Flowᵢₙₛₚ) is predefined freely and the inspiratory ventilation pressure (Pᵢₙₛₚ) is obtained correspondingly and can directly or indirectly be influenced by the stimulation in volume-controlled ventilation modes,
the pattern of the inspiratory ventilation pressure (Pᵢₙₛₚ) is predefined during the ventilation and the inspiratory flow rate (Flowᵢₙₛₚ) is obtained correspondingly and can directly or indirectly be influenced by the stimulation.

Besides pressure-controlled ventilation modes (Pressure Control, PC) and volume-controlled ventilation modes (Volume Control, VC), there also are variants of pressure-controlled ventilation modes, such as variants with constant pressure support, variants with a changeover between two different pressure levels in the course of the ventilation, intermittent ventilation with pressure support and variants of volume-controlled ventilation modes, such as volume-controlled ventilation with pressure limitation, intermittent ventilation with pressure support with guarantee of a minute volume. Furthermore, embodiments and variants of supporting ventilation modes are known as well, but their assignment to pressure-controlled and/or volume-controlled ventilation modes is not absolutely unambiguous. For example, volume-controlled ventilation modes may be supplemented with a pressure limitation, so that a volume-controlled and pressure-regulated ventilation is obtained in such a case. A functionality with a so-called “volume guarantee,” often also called “AutoFlow,” yields, in terms of effect, for example, a volume-controlled and pressure-regulated ventilation. Designations as “regulated” ventilation modes are therefore more proper in some variants and in configurations with additional possibilities of setting and functionalities on ventilators. Within the framework of the present invention, the aspects, which are mentioned, explained and/or described in connection with pressure-controlled and/or volume-controlled ventilation modes as well, can also be extrapolated to pressure-regulated and/or volume-regulated variants and constellations, without the fact that pressure-regulated and/or volume-regulated ventilation modes shall be covered as well being always explicitly referred to in the respective connection with pressure-controlled and/or volume-controlled ventilation modes in the description. Such embodiments and variants of supporting ventilation modes include, for example, so-called assisted ventilation modes with spontaneous breathing support.

Below is a list of some ventilation modes in Table 1 and Table 2.

TABLE 1

| VC modes | Volume-controlled ventilation |
|---|---|
| VC-CMV | Volume Control-Continuous Mandatory Ventilation |
| | Continuous volume-controlled ventilation with fixed |
| | inspiration flow |
| VC-SIMV | Volume Control-Synchronized Intermittent Mandatory |
| | Ventilation/Pressure Support |
| | Intermittent, triggered, volume-controlled ventilation with |
| | fixed inhalation flow and allowed spontaneous breathing |
| | during the phase of exhalation |
| VC-AC | Volume Control-Assist Control |
| | Assisted controlled, volume-controlled ventilation with fixed |
| | inhalation flow and backup frequency |
| VC-MMV | Volume Control-Mandatory Minute Volume Ventilation |
| | Volume-controlled ventilation to ensure a mandatory minute |
| | volume |

The breath volume delivered depends in all pressure-controlled ventilation modes on the pressure difference between assistance pressure and PEEP, the lung mechanics (resistance and compliance) and the respiratory drive of the patient.

TABLE 2

| PC modes | Pressure-controlled ventilation |
|---|---|
| PC-CMV | Pressure Control-Continuous Mandatory Ventilation |
| | Continuous pressure-controlled ventilation with allowed |
| | spontaneous breathing (open system) during the entire |
| | breathing cycle |
| PC-BIPAP | Pressure Control-Biphasic Positive Airway Pressure |
| | Intermittent, synchronized, pressure-controlled ventilation |
| | with allowed spontaneous breathing during the entire |
| | breathing cycle and expiratory synchronization |
| PC-SIMV | Pressure Control-Synchronized Intermittent Mandatory |
| | Ventilation |
| | Intermittent, triggered, pressure-controlled ventilation with |
| | allowed spontaneous breathing during the entire breathing |
| | cycle |
| PC-AC | Pressure Control-Assist Control |
| | Assisted controlled, pressure-controlled ventilation with |
| | allowed spontaneous breathing during the entire breathing |
| | cycle and backup frequency |
| PC-PSV | Pressure Control-Pressure Support Ventilation |
| | Pressure-assisted ventilation with guaranteed minimum |
| | frequency (backup frequency) |
| PC-APRV | Pressure Control-Airway Pressure Release Ventilation |
| | Spontaneous breathing under continuously positive airway |
| | pressure with short-term pressure releases |
| SPN- | Spontaneous Continuous Positive Airway Pressure/Pressure |
| CPAP/PS | Support |
| | Spontaneous breathing with continuously positive pressure |
| | level with or without pressure support |
| SPN- | Spontaneous Continuous Positive Airway Pressure/Volume |
| CPAP/VS | Support |
| | Spontaneous breathing with continuously positive pressure |
| | level with or without volume support |
| SPN-PPS | Spontaneous Proportional Pressure Support |
| | Spontaneous breathing with flow- and volume-proportional |
| | pressure support |

Some explanations will be given below in a short form for the ventilation modes listed in Table 1 and Table 2. The term PEEP level (positive end expiratory pressure) designates the pressure level in the lungs at the end of exhalation, i.e., the positive end-expiratory pressure (PEEP).

The ventilation mode VC-CMV provides a continuous, volume-controlled ventilation with an inspiratory flow rate (flow) predefined at a fixed value, which determines the pressure increase. If this flow rate is so high that the breath volume set is reached already before the end of the set inhalation time, there will be an inspiratory pause. The mandatory ventilation strokes are time-controlled and are not triggered by the living being or the patient. The number of mandatory ventilation strokes is determined by the respiration rate.

The ventilation mode VC-SIMV provides an intermittent, triggered, volume-controlled ventilation with spontaneous breathing allowed during the entire breathing cycle. The mandatory ventilation strokes may be triggered by inhalation efforts of the living being or of the patient at the PEEP level. A mandatory ventilation stroke can only be triggered within a “trigger window,” triggered by a triggering (flow trigger) on the basis of the inspiratory flow rate and synchronized with the spontaneous inhalation. This prevents the possibility of an application of a mandatory ventilation stroke during the spontaneous exhalation. When the patient is already inhaling at the beginning of the trigger window and has already inhaled a substantial volume, the ventilator takes this volume into consideration when balancing the introduced and removed quantities of gas. The inspiratory phase is shortened for this during the next mandatory ventilation stroke and the inspiratory pause is prolonged. The inhalation efforts are synchronized by setting the trigger conditions. Inhalation efforts of the living being or of the patient trigger respective pressure-assisted ventilation strokes at the PEEP level in case of pressure support.

The ventilation mode VC-AC provides an assisted controlled, volume-controlled ventilation with inspiratory flow rate (inhalation flow) set at a fixed value and with a backup frequency. Each inhalation effort of the living being or of the patient at the PEEP level triggers a synchronized mandatory ventilation stroke. The time and the number of mandatory ventilation strokes are thus determined by the living being or the patient. The trigger window comprises the exhalation time minus an estimated time for the preceding exhalation. The exhalation time is obtained from the respiration rate and the inhalation time. No later than after the end of the exhalation time, a non-synchronized mandatory ventilation stroke is triggered (backup frequency). The minimum number of mandatory ventilation strokes is determined by the respiration rate.

The ventilation mode VC-MMV provides a volume-controlled ventilation to ensure a mandatory minute volume. MMV behaves like SIMV, but the mandatory ventilation strokes are only administered when the spontaneous breathing is not sufficient and drops below a predefined minimum ventilation. If the spontaneous breathing increases, fewer mandatory strokes are administered. The minimum ventilation is obtained by setting the breath volume and the respiration rate. The maximum number of the mandatory ventilation strokes is determined by the respiration rate. This number is, however, administered only when a sufficient spontaneous breathing is not present. A pressure support can be selected. In case of pressure support, inhalation efforts of the living being or of the patient trigger respective pressure-supported ventilation strokes at the PEEP level. By setting the triggering conditions, the inhalation efforts are synchronized. The time, number and duration of the pressure-supported ventilation strokes are determined by the spontaneous breathing of the living being or of the patient. The pressure support is terminated as soon as the inhalation flow drops below a percentage of the maximum inhalation flow.

The ventilation mode PC-CMV provides a continuous, pressure-controlled ventilation. The mandatory ventilation strokes are time-controlled and are not triggered by the living being or the patient. The number of the mandatory ventilation strokes is determined by the respiration rate.

The ventilation mode PC-BIPAP provides an intermittent, synchronized, pressure-controlled ventilation with allows spontaneous breathing during the entire breathing cycle with expiratory synchronization. The changeover from inspiratory to expiratory pressure level is synchronized with the spontaneous breathing of the living being or of the patient. The mandatory ventilation strokes can be triggered by inhalation efforts of the living being or of the patient at the PEEP level. A mandatory ventilation stroke can only be triggered within a “trigger window,” triggered by a triggering (flow trigger) on the basis of the inspiratory flow rate and synchronized with the spontaneous inhalation. The possibility of applying a mandatory ventilation stroke during the spontaneous exhalation is prevented thereby.

The ventilation mode PC-SIMV provides an intermittent, triggered, pressure-controlled ventilation with allowed spontaneous breathing during the entire breathing cycle. A mandatory ventilation stroke can only be triggered within a “trigger window,” triggered by a triggering (flow trigger) on the basis of the inspiratory flow rate and synchronized with the spontaneous inhalation. The possibility of application of a mandatory ventilation stroke during the spontaneous exhalation is prevented thereby.

In the PC-AC ventilation mode, the ventilator supports a pressure-controlled ventilation with allowed spontaneous breathing during the entire breathing cycle. Inhalation efforts of the living being or of the patient at the PEEP level trigger respective synchronized, pressure-supported ventilation strokes. The time and the number of the mandatory ventilation strokes are thus determined by the living being or the patient. The trigger window for triggering mandatory ventilation strokes comprises the exhalation time minus a protected time for the preceding exhalation. The exhalation time is obtained from the respiration rate and the inhalation time. No later than after the end of the exhalation time, a non-synchronized mandatory ventilation stroke (backup frequency) is triggered. The minimum number of mandatory ventilation strokes is determined by the respiration rate.

In the ventilation mode PC-PSV, the ventilator supports the spontaneous breathing. Inhalation efforts of the living being or patient at the PEEP level trigger respective pressure-supported ventilation strokes. The inhalation efforts are synchronized by setting the trigger conditions. If the respiration rate of the living being or patient is lower than the set backup frequency, there is no spontaneous breathing, and pressure-supported ventilation strokes with the respiration rate are administered in a time-controlled manner.

In the ventilation mode PC-APRV, the ventilator supports the spontaneous breathing with short-term pressure releases. The patient or the living being can breath spontaneously at a high pressure level with settable duration. The ventilator reduces the exhalation pressure to a low pressure level for very short exhalation times. When an AutoRelease functionality is activated, the duration of the pressure release is determined from the curve (time course) of the flow rate during the exhalation. When the AutoRelease functionality is activated, the changeover from an upper pressure level to the lower pressure level is synchronized with the spontaneous breathing of the living being or patient.

In the ventilation mode SPN-CPAP/PS, the ventilator supports the spontaneous breathing with a continuously positive pressure level. A pressure support can be selected In case of pressure support, inhalation efforts of the living being or patient at the PEEP level trigger respective pressure-supported ventilation strokes. The pressure support is terminated as soon as the inhalation flow drops below a percentage of the maximum inhalation flow or the duration of the support exceeds a maximum inhalation time.

In the ventilation mode SPN-CPAP/VS, the ventilator supports the spontaneous breathing with a continuously positive pressure level. A volume support can be selected. In case of volume support, inhalation efforts of the living being or patient at the PEEP level trigger respective volume-supported ventilation strokes. The inhalation efforts are synchronized by setting the trigger conditions. The volume support is terminated as soon as the inhalation flow drops below a percentage of the maximum inhalation flow or the duration exceeds a maximum inhalation time.

In the ventilation mode SPN-PPS, the ventilator intensifies the spontaneous breathing of the living being or patient proportionally to the patient's effort. If the patient is breathing heavily, the ventilator responds with a high pressure support. If the patient's breathing is shallow, the ventilator responds with a weak pressure support. In the absence of spontaneous breathing, the mechanical support is absent as well. The degree of support can be set in case of PPS. A functionality of a volume guarantee, often also called AutoFlow, should also be mentioned as another possibility of setting on a ventilator. If such a setting is activated, the ventilator doses with a decreasing flow rate. Peaks of the ventilation pressure can be avoided in this manner. The ventilator delivers additional quantities of breathing gas as soon as the patient also breathes spontaneously.

The ventilation modes can only be configured for the individual use at the living being by means of ventilation parameters that can be set by the user.

Tables 3 and 4 below show some parameters, which can be set in volume-controlled or pressure-controlled ventilation modes.

TABLE 3

| | | VC- | CV- | VC- | VC- |
|---|---|---|---|---|---|
| | | SIMV | CMV | AC | MMV |
| Inspiratory oxygen | FiO₂ | X | X | X | X |
| concentration | | | | | |
| Tidal volume | VT | X | X | X | X |
| Duration of inhalation | Ti | X | X | X | X |
| Respiratory rate (RR) | f | X | X | X | X |
| Flow rate (Flow) | v′ | X | X | X | X |
| Maximum pressure | P_{MAX} | X | X | X | X |
| Positive end-expiratory | PEEP | X | X | X | X |
| pressure | | | | | |
| Pressure support | ΔP_{SUPP} | X | | | X |

TABLE 4

| | | PC- | PC- | PC- | PC- | PC- | PC- |
|---|---|---|---|---|---|---|---|
| | | SIMV | BIPAP | AC | CMV | APRV | PSV |
| Inspiratory oxygen | FiO₂ | X | X | X | X | X | X |
| concentration | | | | | | | |
| Tidal volume | VT | X | | X | X | | X |
| Duration of inhalation | Ti | X | X | X | X | | |
| Respiratory rate (RR) | f | X | X | X | X | | X |
| Flow rate (Flow) | | X | X | X | X | | X |
| Maximum pressure | P_{MAX} | X | X | X | X | X | X |
| Inhalation pressure | P_{INSP} | X | X | X | X | X | |
| Positive end.- | PEEP | X | X | X | X | | X |
| expiratory pressure | | | | | | | |
| Pressure support | ΔP_{SUPP} | X | X | | | | |
| Max. duration of | Ti_{MAX} | | | | | | X |
| inhalation | | | | | | | |

Stimulations during the exhalation or during phases of exhalation can be avoided by detecting times of a beginning of phases of inhalation and phases of inhalation with durations of the stimulation, which are adapted to the duration of the phases of inhalation.

Information which arises from the knowledge of the ventilation mode in conjunction with ventilation parameters selected and set during the ventilation with the corresponding ventilation mode employed can be checked, for example, for plausibility in connection with data or signals of pressure sensors and/or flow rate sensors over the course of time or be synchronized with one another and be taken into consideration for the stimulation and for the time course (timing) of the stimulation.

For example, information provided on changes in the inspiratory ventilation pressure (Pᵢₙₛₚ) in volume-controlled ventilation modes can thus be used as feedback in relation to the effect of the stimulation for subsequent adaptations of the stimulation.

For example, information provided on changes in the inspiratory flow rate (Flowᵢₙₛₚ) in pressure-controlled ventilation modes can thus be used as feedback in relation to the effect of the stimulation for subsequent adaptations of the stimulation.

For example, information provided on changes in the inspiratory flow rate (Flowᵢₙₛₚ) in volume-controlled ventilation modes can thus be used for the chronological synchronization of stimulation and breathing or ventilation.

The information on changes in the inspiratory flow rate (Flowᵢₙₛₚ) may comprise here typical patterns of the inspiratory flow rate (Flowᵢₙₛₚ) as well as of an inspiratory volume determined herefrom by means of integration over time over the time course before, during and after the effect of the stimulation on the living being.

For example, information provided on changes in the inspiratory ventilation pressure (Pᵢₙₛₚ) in pressure-controlled ventilation modes can thus be used for a chronological synchronization of stimulation and breathing or ventilation. The information on changes in the inspiratory ventilation pressure (Pᵢₙₛₚ) may comprise here typical patterns of the ventilation pressure (Pᵢₙₛₚ) in the time course before, during and after the effect of the stimulation on the living being.

In ventilation modes with volume-controlled ventilation, information concerning an inspiratory flow rate (Flow) are used to make a time course (timing) of the stimulation synchronous with ventilation cycles. The signal of the flow rate is characteristic in volume-controlled ventilation, and there is a steep rise in the signal of the flow rate at the start of the inhalation, and there is a steep drop of the signal of the inspiratory flow rate (Flow) by the end of the inhalation. Since the ventilator seeks to maintain the flow rate (Flow) extensively constant with the control of the ventilation in case of a volume-controlled ventilation, the flow rate and hence also information or signals that indicate inspiratory flow rates cannot be considered to be able to be used to detect an effect of a stimulation on the diaphragm. Contrary to this, the ventilation pressure and hence also information or signals that indicate inspiratory ventilation pressures can be used to detect activation or activities of the diaphragm. This can be made visible for a user, for example, in a presentation of the ventilation pressure in a display on a ventilator, as well as based on an increase in the determined and displayed tidal volume (VT). By contrast, the signal of the flow rate is of a lesser central interest for a display on the ventilator during volume-controlled ventilation, and the signal of the flow rate is rather significant for the synchronization between the ventilator and the stimulation device.

According to a first aspect of the present invention, provided information and data and/or provided measured values in relation to a state of ventilation of a living being or in relation to an operating state of a ventilator are used in a process according to the present invention for carrying out a stimulation of the nervous system for a ventilation of a living being. The information is provided here in the form of at least one piece of information (information). The carrying out of the stimulation as well as an adaptation of the carrying out of the stimulation can be configured on the basis of the information. Information is defined as data and measured values provided in the context of the present invention, which can have an information content concerning an operating state of a ventilator as well as an information content concerning a state of ventilation of a living being.

These include, for example, measured values of sensors, such as pressure sensors and flow rate sensors.

The sensors may be configured here as elements of the ventilator as well as as elements of a measuring device or as elements of a stimulation device.

Ventilators may be configured, for example, as an intensive-care ventilator, as an emergency ventilator, as a transport ventilator, as a neonatal ventilator or as an anesthesia apparatus.

Information concerning a state of ventilation of a living being may furthermore be provided by devices that are configured to detect measured values, parameters or additional data in relation to a state of health, especially a state of the lungs of a living being as well as the state of ventilation.

These include, for example, devices for an imaging analysis or diagnosis, devices for a blood analysis or diagnosis, devices for a blood gas analysis or diagnosis, devices for determining an oxygen saturation or an oxygen concentration in the blood, devices for determining an oxygen concentration in breathing gases, devices for carbon dioxide saturation or carbon dioxide concentration in the blood, and devices for an invasive or non-invasive blood pressure measurement. Devices for an imaging analysis or diagnosis are, for example, devices for an electrical impedance tomography or EIT systems, devices for a magnetic resonance imaging (MRI), devices for computed tomography (CT), and devices for ultrasound imaging.

The provision of information in relation to a state of ventilation of a living being or in relation to an operating state of a ventilator may take place, for example,
by means of a data input by a user,
by means of a data interface directly from the ventilator,
by means of a data interface to a data network with coupling with a hospital management system or patient data management system, and
by means of an analysis of provided information, data or measured values.

Data inputs by the user may be used to make available information concerning an operating state of a ventilator. Operating states of the ventilator are characterized especially by settings or parameterizations of the ventilators. These include especially the setting selected by the user, which determines whether the ventilator is used in an operating state with a pressure-controlled ventilation mode or in an operating state with a volume-controlled ventilation mode. Furthermore, it is also possible to select types of ventilation modes, such as ventilation modes for ventilating adults or newborns (neonatal ventilation modes), especially ventilation modes with the use of high-frequency ventilation (HF ventilation) for such settings or parameterizations, which can be selected by the user.

Data inputs by the user may, furthermore, be used to make available personal information, such as general and physical constitution, age, height, body weight, sex, clinical picture, course of the disease, past medical history, diagnoses, findings for a patient or living being.

Data interfaces to a ventilator may, furthermore, be used to make available personal information, such as general and physical constitution, age, height, body weight, sex, clinical picture, course of the disease, past medical history, diagnoses, findings for a patient or living being.

Data interfaces to a data network, e.g., to a hospital management system or patient data management system, may, furthermore, be used to make available personal information, such as general and physical constitution, age, height, body weight, sex, clinical picture, course of the disease, past medical history, diagnoses, findings for a patient or living being.

Data interfaces to a ventilator may provide information and/or measured values on an operating state or an operating situation of the ventilator.

Information that indicates an operating state or an operating situation of a ventilator may be derived, for example, from measured values such as measured pressure values and/or pressure/time courses (curves) of the inspiratory and/or expiratory ventilation pressure, measured flow rate values (Flow), flow rate/time courses, measured volume values, volume/time courses. The measured values may be obtained here on the basis of pressure sensor mechanisms and/or flow rate sensor mechanisms arranged at or associated with the ventilator.

It is, however, also possible in alternative embodiments to use a pressure sensor mechanism and/or flow rate sensor mechanism arranged outside of or associated with the ventilator, for example, in a measuring device or as elements of a stimulation device for the measurement-based detection of the inspiratory and/or expiratory ventilation pressure, of pressure/time courses of the inspiratory and/or expiratory ventilation pressure, of measured flow rate values (Flow), of flow rate/time courses, of measured volume values and/of or volume/time courses.

Data interfaces to a measuring device, to a stimulation device or to a ventilator can provide information and/or measured values concerning the situations of the ventilation of the living being, such as measured pressure values and/or pressure/time courses of the inspiratory and/or expiratory ventilation pressure, measured flow rate values (Flow), flow rate/time courses, measured volume values, and volume/time courses.

Data interfaces to a hospital management system or patient data management system may be used to make available personal information, such as general and physical constitution, age, height, body weight, sex, clinical picture, course of the disease, past medical history, diagnoses, findings for a patient or living being.

The following list comprises, without laying a claim to completeness, a group comprising information on ventilation settings, alarms in connection with operating states of a ventilator, which may form a plurality of embodiments each in itself as well as in combination with one another:
type of a ventilation mode, such as volume-controlled or pressure-controlled ventilation mode,
types of ventilation modes for adults, children, newborns, premature infants (neonatal ventilation modes, HF ventilation modes),
manner of feeding breathing gases, such as endotracheal tube, non-invasive breathing mask, tracheostomy,
ventilation settings or parameters of the ventilator, such as
   ventilation frequency (RR),
   tidal volume (VT),
   minute volume (MV),
   inhalation to exhalation ratio (I:E ratio),
   breath volume,
   airway pressure,
   inspiratory ventilation pressure,
   expiratory ventilation pressure,
   positive end-expiratory pressure (PEEP),
time of a start or of an end of the rising flank of an inspiratory ventilation pressure,
time of a start or of an end of the plateau phase of the inspiratory ventilation pressure,
inspiratory oxygen concentration (FiO₂),
expiratory carbon dioxide concentration (etCO₂),
setting limits, alarm generations, alarm settings for alarm generations by the ventilator with upper limits and lower limits
   of an expiratory minute volume,
   of an airway pressure,
   of an inspiratory O₂ concentration,
   of an end-expiratory CO₂ concentration,
   of a volume monitoring,
   of an upper limit of a tachypnea monitoring,
   of a time range monitoring of an apnea alarm time,
reports, warnings or alarm generations in case of exceeding/undershooting upper/lower limits and predefined time ranges.

In addition, the at least one piece of information may also comprise information in relation to properties of a living being or patient, such as general and physical constitution, age, height, body weight, sex, clinical picture, course of the illness, past medical history, diagnosis, and findings.

A stimulation signal is generated according to the present invention for carrying out the stimulation for influencing the nervous system of a living being on the basis of at least one piece of information or of at least one of the information which indicates a state of a breathing or of a ventilation and/or an operating state of a ventilator.

Stimulation and influencing of the phrenic nerve is carried out in an especially preferred embodiment of the process with the carrying out of the stimulation with influencing of the nervous system of the living being by means of the stimulation signal. The stimulation and the influencing of the phrenic nerve are controlled and synchronized here with the breathing activity of the living being or with a triggering of a breathing activity or ventilation of the living being on the basis of the at least one piece of information. The at least one piece of information indicates a type of a ventilation mode. The control and synchronization of the stimulation takes place such that if at least one piece of information indicates that the ventilation is being carried out in a volume-controlled ventilation mode, a piece of information, which indicates a flow rate or a volume, is also included.

In a volume-controlled ventilation mode, the time course of the signal of the flow rate is especially suitable for basing the carrying out of the stimulation on it, because this curve has an essentially rectangular configuration in case of volume-controlled ventilation and the signal course of the ventilation pressure will then arise from this in alternation with flow resistances in the ventilation system and with the situation of the lungs of the patient.

The control and synchronization of the stimulation take place such that if the at least piece of information indicates that the ventilation is being carried out in a pressure-controlled ventilation mode, a piece of information that indicates a ventilation pressure and/or a piece of information that indicates a flow rate or a volume is also included.

In case of a pressure-controlled ventilation mode, both the time course of the signal of the ventilation pressure and the time course of the signal of the flow rate are suitable for basing the carrying out of the stimulation on it, because both curves have an essentially rectangular configuration in case of pressure-controlled ventilation at the beginning of the inhalation phase.

In the course of the ventilation and in the course of the stimulation, an adaptation of the manner of the stimulation with influencing of the nervous system on the basis of changes in the information can take place in the course of the ventilation in a preferred embodiment.

Embodiments show the manners in which the carrying out of the stimulation with influencing of the nervous system of a living being can be configured. Embodiments show how a selection, an activation or a deactivation of modes of operation can be carried out on a stimulation device by means of a stimulation signal
for operation in a first mode of operation of a follow mode and
for an operation in a second mode of operation of a lead mode.

Other embodiments show how the ventilation of the living being can be carried out in an operating state with a pressure-controlled ventilation mode and how the first mode of operation can be activated by the stimulation signal with a follow mode at the stimulation device or how the second mode of operation can be activated with a lead mode at the stimulation device.

Other embodiments show how the ventilation of the living being can be carried out in an operating state with a volume-controlled ventilation mode on the basis of the at least one piece of information and how the first mode of operation can be activated now with a follow mode at the stimulation device or how the second mode of operation can be activated with a lead mode at the stimulation device.

The different aspects, peculiarities and differences between the “follow mode” and the “lead mode” will now be explained in more detail below on the basis of embodiments.

The stimulation or the control of the stimulation device follows in the follow mode the ventilation pattern, which is predefined by the ventilator or the inhalation trigger of the patient or living being. Information from sensors, preferably pressure sensors and/or flow rate sensors, are needed for carrying out the stimulation in order to determine activities of the ventilator or also of spontaneous activities elicited by the living being in the course of the ventilation. Activities of the ventilator and spontaneous activities are influenced only slightly in this follow mode.

The ventilator follows in the lead mode with the configuration of the course of ventilation of the stimulation, which is predefined by the stimulation device or by the inhalation trigger of the living being. Usual functions of a triggering of ventilation strokes can be used for this at the ventilator in order to respond to stimulated inhalation efforts of the living being on the part of the ventilator. These include, for example, flow triggers as well as pressure triggers.

An embodiment, in which an activation of the stimulation signal takes place after the onset of an inhalation by the living being, after an inhalation trigger or after the initiation of a phase of inhalation by the ventilator, can thus be configured, for example, in the follow mode. Such an inhalation trigger makes it possible to detect a beginning of an inhalation phase or a detection of an incipient inhalation activity elicited by the living being. The manner of detection may be based on a detection on the basis of an exceeding of a threshold value of the inspiratory ventilation pressure or of the inspiratory flow rate.

Detection of an end of a phase of inhalation can be based on a detection of an undershooting of a threshold value of the inspiratory ventilation pressure or of the inspiratory flow rate. The flow rate remains now in the positive range, i.e., the reversal of the direction of the breathing gas flow between inhalation into the directly following exhalation has not yet occurred. A first mode of operation according to such an embodiment can be called a so-called follow mode. The stimulation follows in the follow mode the ventilation pattern in real time, and this ventilation pattern is predefined by the ventilator or by the inhalation trigger of the living being.

In the follow mode, the stimulation follows the rise of the inspiratory flank of the ventilation in real time in a synchronized manner and is terminated immediately as soon as the inspiratory pause begins and always when the ventilator has stopped the feed of inspiratory quantities of breathing gases. Then, there will be no stimulation for activation of inhalation efforts of the living being or patient by means of stimulation during the inspiratory pause. Availability of a signal of a sensor is necessary for the stimulation in the follow mode. This signal makes it possible to detect which breathing activity has been elicited by the living being and which ventilation activity has been elicited by the ventilator. Signals of a pressure sensor as well as additionally also signals of a flow rate sensor are preferably available. Such a sensor mechanism may advantageously be configured as a combination of a so-called “flow/pressure sensor mechanism.” Such a sensor mechanism may be a part of a ventilator and also a part of a measuring device additionally introduced into the breathing gas feed line and/or discharge line leading to/from the living being. The follow mode may be implemented for both pressure-controlled ventilation and volume-controlled ventilation in the following manner:

For the pressure-controlled ventilation, either a rise over time of the time course of the flow rate is determined by a flow rate sensor mechanism intended for this purpose and configured in a suitable manner or it is provided by means of a data exchange from the ventilator to the stimulation device to detect an end of a phase of inhalation.

For the volume-controlled ventilation, either a pressure rise is provided by a pressure sensor mechanism intended for this purpose and configured in a suitable manner for this or it is made available by means of a data exchange from the ventilator to the stimulation device to detect an end of the phase of inhalation. The following procedure is used to illustrate the data exchange for a stimulation in the follow mode during a pressure- and/or volume-controlled ventilation. The stimulation device follows the ventilation rhythm predefined by the ventilator in the follow mode.

### Follow Mode

Predefined values provided by the ventilator:
   ventilation rhythm with respiratory rate (RR=respiratory rate) and inhalation to exhalation ratio (I:E ratio)
      during volume-controlled ventilation: Volume and flow rate,
      during pressure-controlled ventilation: Ventilation pressure,
Contribution of the stimulation device during volume-controlled ventilation
   The stimulation device uses data that indicate an inspiratory flow rate, for example, data or measured values of an inspiratory flow rate sensor, for detecting an exceeding of a flow rate threshold value at the start of a rectangular curve of the ventilation pressure of the phase of inhalation and for detecting an undershooting of another flow threshold value for terminating the phase of inhalation with the rectangular curve of the flow rate.
Result of the stimulation during volume-controlled ventilation:
The contribution of the stimulation device leads to a reduction of the airway pressure compared to a carrying out of the ventilation without an application of stimulation at the beginning of phases of inhalation.
Contribution of the stimulation device during pressure-controlled ventilation:
The stimulation device uses data that indicate an inspiratory ventilation pressure, for example, for example, data or measured values of an inspiratory pressure sensor, for detecting an exceeding of a pressure threshold value at the start of a rectangular curve of the ventilation pressure of the phase of inhalation and for detecting an undershooting of another pressure threshold value at the end of the phase of inhalation with the rectangular curve of the ventilation pressure. In an alternative embodiment, the stimulation device may use data that indicate an inspiratory flow rate in order to detect the start of the phase of inhalation and to determine the end of the phase of inhalation on the basis of a criterion.

Such a criterion may be configured, for example, as a so-called “cycling off criterion,” which describes a state in which a drop in the flow rate detected currently by measurement to, for example, about 15%-25% of a value of a maximum inspiratory flow rate is present. This corresponds approximately to a situation with lungs filled with the desired quantity of breathing gas during the progression of the phase of inhalation.
Result of a stimulation during pressure-controlled ventilation:
The contribution of the stimulation device leads to an increase in the quantity of inspiratory gas quantities compared to carrying out the ventilation without an application of a stimulation at the beginning of phases of inhalation.

According to a preferred embodiment, a signal of a flow rate sensor and/or a signal of a pressure sensor can be used during the carrying out of the ventilation in a volume-controlled ventilation mode in combination with a stimulation of the nervous system to trigger a breathing effort of the living being in a first mode of operation in the follow mode.

According to a preferred embodiment, a signal of a flow rate sensor and/or a signal of a pressure sensor can be used as an inhalation trigger during the carrying out of the ventilation in a pressure-controlled ventilation mode in combination with a stimulation of the nervous system to trigger an inhalation effort of the living being in a first operating mode in the follow mode.

Another embodiment can be formed, in which an activation of the stimulation signal takes place prior to the onset of an inhalation by the living being or detection of an inhalation trigger elicited by the living being and after the initiation of a phase of inhalation by the ventilator. A second mode of operation according to such an embodiment may be called a so-called lead mode. The ventilator follows in the lead mode with the configuration of the ventilation process of the stimulation, which is predefined by the stimulation device or by the inhalation trigger of the living being. Usual functions of a triggering of ventilation strokes can be used for this at the ventilation direction to respond to stimulated breathing effort of the living being on the part of the ventilator. These include, for example, flow triggers as well as pressure triggers.

The following process is used to illustrate the data exchange for a stimulation in the lead mode during a pressure- and/or volume-controlled ventilation. The stimulation device predefines a ventilation rhythm in the lead mode.

### Lead Mode

Predefined values by the stimulation device:
   Ventilation rhythm with ventilation frequency (RR=respiratory rate) and inhalation to exhalation ratio (I:E ratio)
      volume and flow rate in case of volume-controlled ventilation,
      ventilation pressure in case of pressure-controlled ventilation,
Contribution of the ventilator during volume-controlled ventilation:
   The ventilator detects the activation of the stimulation at the beginning of an activation of a feed or delivery of inspiratory flow rates of breathing gases to the patient or living being. The detection of activation or the detection of triggering may take place both by a data exchange between the ventilator and the stimulation device or on the basis of signals or data of a flow rate sensor.
   The ventilator detects the deactivation of the stimulation at the end of the feed or delivery of inspiratory flow rates of breathing gases. The detection of the deactivation or detection of the cycling-off state can take place both by a data exchange between the ventilator and the stimulation device or on the basis of signals or data of a pressure sensor, which indicate a pressure rise of the ventilation pressure at the end of the activation phase of the respiratory muscles.
Contribution of the ventilator during volume-controlled ventilation:
   The ventilator detects the activation of the stimulation at the beginning of an activation of provision of an inspiratory pressure level to the patient or living being. The detection of the activation or detection of the triggering may take place both by a data exchange between the ventilator and the stimulation device or on the basis of signals or data of a flow rate sensor.
   The ventilator detects the deactivation of the stimulation for termination of the provision of the inspiratory pressure level. The detection of the deactivation or detection of the cycling off state can take place both by a data exchange between the ventilator and the stimulation device or on the basis of signals or data of a flow rate sensor.

According to a preferred embodiment, a signal of a flow rate sensor and/or a signal of a pressure sensor can be used as an inhalation trigger during a carrying out of the ventilation in a volume-controlled ventilation mode in combination with a stimulation of the nervous system to trigger a breathing effort of the living being in a first mode of operation in the lead mode.

According to a preferred embodiment, a signal of a flow rate sensor and/or a signal of a pressure sensor can be used as an inhalation trigger during the carrying out of the ventilation in a pressure-controlled ventilation mode in combination with a stimulation of the nervous system for triggering a breathing effort of the living being in a first mode of operation in the lead mode.

The inhalation trigger can be triggered now during a ventilation by a ventilator by an exceeding of a threshold value of an inspiratory flow rate (Flowᵢₙₛₚ) or by an exceeding of a threshold value of an inspiratory ventilation pressure (Pᵢₙₛₚ). In an alternative embodiment, the inhalation trigger may be elicited by means of surface electromyography (sEMD).

Preferred embodiments of the process can be configured, in which information, data or measured values are used to activate the stimulation signal for the stimulation with influencing of the nervous system of the living being depending on the operating state or operating situation of the ventilator in combination with the respective activated mode of operation of the stimulation device. It is essential for an application of the stimulation that the stimulations bringing about a muscle activity for initiating an inhalation must have been clearly and reliably terminated prior to a possible onset of the exhalation or of an exhalation effort in order for a stimulation to be avoided in any case with certainty during an exhalation. A longer duration may be selected in the lead mode for the stimulation. This leads to possibilities of variations concerning the form of the stimulations, for example, with a ramp-like rise.

Unlike in the case of the lead mode, it is necessary in the follow mode to wait for the activity of the ventilator for initiating the phase of inhalation, so that only a shorter time interval is available for configuring the ramp-like rise in the follow mode compared to the lead mode. In addition, the amplitude of the stimulation pulse can be selected to be smaller than in the follow mode due to the availability of the longer duration of an action of the stimulation, The time window in which the stimulation can be activated without the stimulation being at risk of still reaching the next phase of exhalation is shorter at higher ventilation frequencies than at lower ventilation frequencies. It follows from this for a comparison of the follow mode and the lead mode that unlike the follow mode, the lead mode can also be used especially advantageously at high ventilation frequencies, i.e., for example, at ventilation frequencies (RR) above 15 breath cycles per minute.

The beginning of the stimulations can ideally be selected in the lead mode at times in the course of the ventilation at which the phases of exhalation are terminated. Such a termination of the phase of exhalation can be derived, for example, from the ventilation regimen with the ventilation mode, ventilation frequency (RR=respiratory rate), inhalation to exhalation ratio (I:E ratio) or from a time course of the flow rates according to the amplitude, direction and times of the reversal of the flow direction, with inspiratory flow rate (Flowᵢₙₛₚ), expiratory flow rate (Flowₑₓₛₚ), as well as patient flow rate (Flow_{Pat}), which can be detected by measurement, for example, by means of a flow rate sensor or a plurality of flow rate sensors.

A combination of signals in the time course of the flow rate with information on the ventilation regimen with ventilation mode, ventilation settings (RR, I:E ratio) may also be configured for a synchronization of the lead mode in such a manner that the stimulation takes place as with a type of “pre-triggering.” Such a “pre-trigger,” i.e., the application of the lead mode, can bring about a synchronous activation of the stimulation with a certain time interval before the inhalation triggerings initiated in the course of the ventilation in interaction between the ventilator and the patient. The inhalation triggerings and their rhythm over time can be determined by regular observation of the ventilation curve from times of mandatorily predefined start times of the phase of inhalation, as well as from inhalation triggerings initiated by means of flow triggers or pressure triggers of the patient, taking into account information on ventilation regimens with the ventilation mode, ventilation frequency (RR=respiratory rate), and inhalation to exhalation ratio (I:E ratio).

The application of the stimulation results especially in the lead mode in the advantage of a reduction or avoidance of the number of peak values of the ventilation pressure.

According to a preferred embodiment, signals of a flow rate sensor and/or a signal of a pressure sensor can be used as inhalation triggers for triggering a breathing effort of the living being in a second mode of operation in the lead mode during the carrying out of the ventilation in a volume-controlled ventilation mode in combination with a stimulation of the nervous system.

Preferred embodiments of the process can be configured, in which information, data or measured values are used for activating the stimulation signal for the synchronization of the stimulation or of the stimulation device and the ventilator depending on the operating state or the operating situation of the ventilator in combination with the respective activated mode of operation of the stimulation device.

It is advantageous for the synchronization in both pressure-controlled and volume-controlled ventilation modes if a flow rate sensor or signals or data, which indicate flow rates or flows to the patient, are available. For example, a start of a phase of inhalation can take place in an especially advantageous embodiment on the basis of a threshold value comparison of the signal of the flow rate sensor and a termination can take place on the basis of the cycling-off criterion. Depending on the ventilation mode, flow rate sensors are then used for the application of the threshold value comparison in case of volume-controlled ventilation and pressure sensors are used in case of pressure-controlled ventilation. Depending on the ventilation mode, flow rate sensors are then used for the application of the cycling-off criterion in case of volume-controlled ventilation and pressure sensors are used in case of pressure-controlled ventilation.

The at least one piece of information, which indicates which ventilation mode is currently activated, may be provided, for example, as a data input by means of a manual input or in a data exchange from the ventilator. The data input may preferably be configured by means of an application interface (GUI, keyboard, touchpad) at the stimulation device. The data exchange between the stimulation device and the ventilator may be configured by means of data interfaces (USB, Ethernet) and data lines or also in a wired or wireless manner in a network linking system (PAN, LAN, WLAN, Bluetooth).

If the information indicates in a preferred embodiment that the ventilation takes place with a ventilation mode of a volume-controlled ventilation, measured values of an inspiratory flow measurement are included during the carrying out of the stimulation. This inclusion is used, on the one hand, to detect a beginning of the inhalation, and, on the other hand, also the determination of the time during the inhalation at which the desired quantities of breathing gases have flowed into the lungs of the living being or patient. The stimulation shall not then be continued beginning from this time in order to avoid a possible hyperinflation of the lungs.

If the information indicates in a preferred embodiment that the ventilation takes place with a ventilation mode with pressure-controlled ventilation, then measured values of an inspiratory ventilation pressure are included during the carrying out of the stimulation.

This inclusion is used, on the one hand, to detect a beginning of the inhalation, and, on the other hand, also to determine the time during the inhalation at which a desired pressure level is present in the lungs of the living being or patient. The stimulation shall not then be continued beginning from this time in order to avoid a possible hyperinflation of the lungs.

For an application of the stimulation device in the lead mode, the ventilator is preferably equipped with a flow rate sensor mechanism and a pressure-measuring sensor mechanism in order to detect the stimulation. This is made possible by means of a usual trigger detection at the ventilator, as it is also used for detecting spontaneous breathing activities, because the breathing initiated by means of muscle stimulation is highly comparable to spontaneous breathing activity.

Preferred embodiments of the process can be configured, which make possible an evaluation of the effect of the stimulation. A quantitative evaluation of the effect of the stimulation can be carried out, for example, by the volumes, which are initiated by spontaneous breathing efforts by means of stimulation, are compared to the volumes that were mandatorily added by the ventilator during the breath. A quotient, which can represent an indicator of the quantity of the stimulation, can be formed from this. A quantitative evaluation of the effect of the stimulation, i.e., of a percentage of effort or breathing activity or work of breathing initiated by muscle activity relative to the overall effect of breathing and ventilation of a living being, can be carried out on the basis of deviations in the time course of the flow rate (Flow), for example, in case of volume-controlled ventilation modes as well. A qualitative evaluation of the effect of the stimulation, i.e., of a percentage of effort or breathing activity or work of breathing initiated by muscle activity relative to the overall effect of breathing and ventilation of a living being, can be carried out on the basis of deviations in the time course of the ventilation pressure, for example, in case of volume-controlled ventilation modes as well.

Breathing cycles with and without stimulations are necessary for the examination of the deviations. The pressure and/or flow rate sensor mechanisms of the ventilator can be used in the lead mode to detect the stimulations and hence also the corresponding effects on the measured values and time courses of the pressure values and/or flow rates.

For an evaluation of the effect by means of the stimulation device, it is necessary and advantageous if the stimulation device has available or is provided with at least one piece of information on the operating state of the ventilator and/or on the state of the ventilation, e.g., regarding whether the ventilator is operating in a pressure-controlled ventilation mode or in a volume-controlled ventilation mode. Another possibility of configuring a checking of the effect of the stimulation can be configured by a sensor array for surface electromyography (sEMD), which is arranged in the region of the abdomen on the skin surface of a patient. It is thus possible to detect by measurement a degree of activity of the muscle, muscle groups or muscles activated before by means of stimulation.

Preferred embodiments of the process may be configured, which make possible a switching from a stimulation in the follow mode to a stimulation in the lead mode in the course of the ventilation. Such a switching may be configured, for example, as an iterative transition, during which the time interval of the start time of the stimulation is increased continuously incrementally over a sequence of ventilation cycles in relation to the start time of the inhalation phase determined or estimated from the observations and from information on the basis of signal observations of flow rates, ventilation pressure and information on the ventilation rhythm with ventilation frequency (RR=respiratory rate) and the inhalation to exhalation ratio (I:E ratio). Triggering of a stimulation during time phases of the exhalation can thus be prevented by means of signal observations of flow rates and ventilation pressure and a reliable transition can thus be formed from a stimulation in the follow mode into a stimulation in the lead mode. Another possibility of a changeover from the follow mode into the lead mode can be formed by initiating a stimulation in the lead mode in a manner of a “test maneuver” during a stimulation in the follow mode—instead of an incremental approximation—at selected times, which were estimated or selected randomly before from signal observations of flow rates, ventilation pressure and information on the ventilation rhythm with ventilation frequency (RR=respiratory rate) and inhalation to exhalation ratio (I:E ratio). The success of the “test maneuver” can then be monitored and evaluated by signal observations of flow rates, ventilation pressure in conjunction with information on the ventilation rhythm with ventilation frequency (RR=respiratory rate) and inhalation to exhalation ratio (I:E ratio) and the stimulation can be terminated, and the stimulation can be continued in the follow mode or with a changeover to the lead mode.

Preferred embodiments of the process can be configured, which make possible an automatic detection of the ventilation modes employed by the ventilator. In particular, such embodiments make it possible to determine whether the ventilator is being used in a mode of operation with a pressure-controlled or volume-controlled ventilation mode. An automatic detection of the ventilation mode employed by the ventilator may take place, for example,
by means of an analysis of time courses of pressures and/or flow rates with comparison to time courses typical of pressure-controlled or volume-controlled ventilation modes, and
by carrying out a maneuver.

An analysis to determine which of the signals or of the signal courses (pressure, flow rate) is subject to smaller fluctuations over time can then be used to identify the “leading variable” during the carrying out of the ventilation, i.e., the pressure or the flow rate.

A suitable maneuver for an automatic or automated detection of the ventilation mode used by the ventilator can be configured, for example, by means of a superimposition of a disturbance to the ventilation. Thus, influencing of the ventilation can be provoked by increasing the airway resistance or by a closure of the airways from time to time by means of a so-called occlusion. Thus, an increase in the airway resistance leads to a reduction of the flow rate during pressure-controlled ventilation. An increase in the airway resistance thus leads to an increase in the ventilation pressure in case of volume-controlled ventilation. Reduction of the airway resistance brings about said effects in an opposite manner. Such maneuvers are and can usually be carried out by means of the ventilator, and it should be noted in this case that a data exchange is carried out between the stimulation device and the ventilator with exchange of the information on whether the ventilator is currently being used in a mode of operation with a pressure-controlled or volume-controlled ventilation mode, as against giving preference to a provocation of a maneuver at the ventilator by the stimulation device. Another possibility of configuring a suitable maneuver would be a generation of a signal superimposition with the effect of an additive increase in the muscle effort or of the work of breathing or breathing activity or of a subtractive reduction of the muscle efforts.

An increased muscle effort leads under pressure-controlled ventilation to an increase in the inspiratory flow rate and to a reduction of the airway pressure during volume-controlled ventilation. Such a maneuver can be carried out during the phase of inhalation of the ventilator with a short duration, for example, for a duration of <0.5 sec. The beginning of the phase of inhalation can take place preferably with a flow rate sensor, which is then configured, as was already described before, as an element of a measuring device, which is arranged in or at the inspiratory gas feed to the living being. Such a flow rate sensor is advantageous for the synchronization between the ventilator and the stimulation or between the carrying out of ventilation supported with stimulations in both the follow mode and in the lead mode and is helpful for a robust embodiment of the carrying out.

Other embodiments can show configurations of how settings for ventilation settings or ventilation parameters on the ventilator can affect the configuration of the mode of operation of the stimulation device.

For example, the pressure/time course of the ventilation pressure with parameters such as the ventilation frequency (RR), tidal volume (VT), minute volume (MV), inhalation to exhalation ratio (I:E ratio), airway pressure, tidal volume, time of a beginning of the rising flank of the inhalation pressure, time of an end of the rising flank of an inhalation pressure, time of a beginning of the plateau of the inspiratory ventilation pressure, time of an end of the plateau of the inspiratory ventilation pressure can thus be used as a database, whether the stimulation of the nervous system takes place with triggering of a breathing effort of the living being in a mode of operation in the lead mode or in the follow mode.

Other embodiments show configurations of how information on the living being, such as general and physical constitution, age, height, body weight, sex, clinical picture, course of the disease, past medical history, diagnoses, findings may be available and be used, whether the stimulation of the nervous system with triggering of a breathing effort of the living being is implemented in a mode of operation in the lead mode or in a mode of operation in the follow mode.

Other embodiments show configurations of how information on the manner of feeding breathing gases, for example, by means of an endotracheal tube, non-invasive breathing mask, nasal cannula or tracheostomy, can be used, whether the stimulation of the nervous system takes place with triggering of a breathing effort of the living being in a mode of operation in the lead mode or in a mode of operation in the follow mode.

Other embodiments show configurations of how alarm generations, alarm reports, warnings, alarm settings or alarm limits can bring about an activation of the stimulation signal for controlling the stimulation. Alarm reports may be used, for example, to switch the stimulation from the lead mode into the follow mode in the presence of an alarm situation or in an error situation until the alarm or error situation is eliminated. The follow mode can thus also represent a kind of a safe “backup mode” for the lead mode.

For example, an alarm generation, which indicates a drop in an oxygen saturation in the blood to below a predefined lower threshold value, can bring about an activation of the stimulation by means of the stimulation signal.

For example, an alarm generation, which indicates an increase in a carbon dioxide concentration in the exhaled gas above a predefined upper threshold value, can thus bring about an activation of the stimulation by means of the stimulation signal.

Thus, an alarm generation, which indicates a drop of a carbon dioxide concentration in the exhaled gas to below a predefined lower threshold value, can, for example, be taken into consideration by means of the stimulation signal during the carrying out and/or configuration of the stimulation. A drop in the carbon dioxide concentration in the exhaled gas may indicate, for example, that the gas exchange in the lungs with feed of oxygen and removal of carbon dioxide is disturbed. One possible cause may be that the ventilation is carried out, for example, with settings of the ventilation frequency (RR) and inhalation to exhalation ratio that can lead to a so-called dead space ventilation, i.e., the gas exchange takes place mostly in the volume of the ventilation tube system and endotracheal tube without appreciable participation of the lungs. As a consequence, there will be a drop in the concentration of carbon dioxide in the exhaled gas.

For example, an alarm generation, which indicates a drop in a minute volume (MV) to below a predefined lower threshold value or an exceeding of the minute volume (MV) above a predefined upper threshold value, can thus bring about an activation of the stimulation by means of the stimulation signal.

For example, an alarm generation, which indicates a drop of the tidal volume (VT) to below a predefined lower threshold value, can thus bring about an activation of the stimulation by means of the stimulation signal.

Other embodiments show configurations of how alarm reports, warnings, alarm settings or alarm limits can bring about a deactivation of the stimulation signal for controlling the stimulation.

For example, an alarm generation, which indicates an exceeding of the inspiratory ventilation pressure or of the airway pressure (PAW) to above a predefined upper threshold value, can thus bring about a deactivation (stop) of the stimulation for this breath or a termination of the carrying out of additional stimulations by means of the stimulation signal.

For example, an alarm generation, which indicates an exceeding of a tidal volume (VT) to above a predefined upper threshold value, can thus bring about a deactivation (stop) of the stimulation for this breath or a termination of the carrying out of additional stimulations by means of the stimulation signal.

For example, an alarm generation, which indicates an exceeding of a minute volume (MV) to above a predefined upper threshold value, can thus bring about a deactivation (stop) of the stimulation for this breath or a termination of the carrying out of additional stimulations by means of the stimulation signal.

Some typical alarm limits are listed in Table 5 below.

TABLE 5

| Parameter | | Description |
|---|---|---|
| Minute volume | MV_{High} | Monitoring of an upper limit value |
| Minute volume | MV_{Low} | Monitoring of a lower limit value |
| Tidal volume | VT_{High} | Setting/monitoring of an upper limit |
| | | value |
| Tidal volume | VT_{Low} | Setting/monitoring of a lower limit |
| | | value |
| Airway pressure | P_{AW} | Monitoring of an upper limit value |
| Ventilation frequency | f, RR | Setting/monitoring of an upper limit |
| | | value |
| Apnea time window | Tₐₚₙ | Monitoring of a time range |
| End-tidal carbon | etCO₂_{High} | Monitoring of an upper limit value |
| dioxide concentration | | |
| End-tidal carbon | etCO₂_{Low} | Monitoring of a lower limit value |
| dioxide concentration | | |

Table 1, Table 2, Table 3, Table 4 and Table 5 are taken from an instruction for use of the applicant in a form adapted to the description in a patent application and represent an exemplary state of the art in connection with ventilation modes, ventilation settings, ventilation parameters, and alarm generations. The device is designated “Infinity Acute Care System—Evita Infinity V500.” The names and designations of the ventilation modes may differ from one another in instructions for use of different ventilators, the distinction as mandatory ventilation modes and ventilation modes with spontaneous breathing support, as well as the distinction as ventilation modes with pressure control or pressure regulation and ventilation modes with volume control or volume regulation arises from the common nature of the terms employed in connection with the use of ventilators, for example, in intensive care.

Embodiments create possibilities for a kind of provision of information as well as for deducing and/or deriving information from data and/or measured data of sensors. The at least one piece of information, which can indicate ventilation parameters, ventilation settings, setting limits, alarm generations, alarm settings for alarm generations by the ventilator with upper limits and lower limits, types of ventilation modes, the type of feeding breathing gases, information on properties of the living being, may be provided, for example, by means of a manual input or, for example, also by means of a data interface.

The at least one piece of information (information) on which type of ventilation modes is activated or on what type of feed of breathing gases is present can be derived, for example, from measured data of a pressure sensor, which is configured to detect an airway pressure.

The at least one piece of information on what type of ventilation modes is activated or what type of feed of breathing gases is present can be derived, for example, from measured data of a flow rate sensor, which is configured to detect quantities of breathing gases fed to and/or removed form the living being.

The at least one piece of information on which type of ventilation modes is activated or which type of feed of breathing gases is present can be derived, for example, from measured data of a flow rate sensor, which is configured to detect quantities of breathing gases fed to and/or removed from the living being, in combination with measured data of a pressure sensor, which is configured to detect an airway pressure.

The process according to the present invention may also be provided as a computer program or computer program product, so that the scope of protection of the present application likewise extends to the computer program product and to the computer program that may include a computer readable storage medium (or media) having computer readable program instructions thereon. The computer program or computer program product with a program code (with computer readable storage medium that can be a tangible device that can retain and store instructions) can make it possible to carry out the process according to the present invention when the program code is executed on a computer, on a processor or on a programmable hardware component.

The solution for accomplishing the object was described above in relation to the process claimed as a first aspect of the present invention. Features, advantages or alternative embodiments mentioned in this connection can likewise be extrapolated to the other objects being claimed and vice versa. The advantages described for the process according to the present invention can be achieved in the same manner or in a similar manner with the device according to the present invention as well as with the described embodiments of the device. Furthermore, the embodiments described and their features and advantages of the process can be extrapolated to the device, just like the described embodiments of the device can be extrapolated to the process. The corresponding functional features of the process are configured here by corresponding concrete modules, especially by hardware components (μC, DSP, MP, FPGA, ASIC, GAL), which may be implemented, for example, in the form of a computer, a processor, a plurality of processors (μC, μP, DSP) or in the form of instructions in a memory area, which can be processed by the processor.

Thus arises another aspect of the present invention, which accomplishes the objects set according to the present invention by a device for carrying out the process for carrying out a stimulation of the nervous system for ventilating a living being.

This device suitable for carrying out the process is configured to carry out the process as well as to also carry out the further steps described in the embodiments each in itself or in a combination.

The device may have to this end a control unit, a data input unit, and a data output unit.

For example, the provision of the information, data, measured values or inputs of a user can thus be carried out by means of the data input unit and the generation and provision of the stimulation signal can be carried out by the data output unit.

The coordination and processing of the information of the data can be carried out, for example, by a control unit.

The control unit has for this purpose elements for data processing, computation and process control, such as microcontrollers (μC), microprocessors (μP), signal processors (DSP), logic modules (FPGA, PLD), memory modules (ROM, RAM, SD-RAM) and combined variants thereof, for example, in the form of an “embedded system,” which are configured jointly with one another and are adapted to one another and are configured by programming to execute the data processing.

The data input unit is configured for providing the information, measured values, data, data sets or for inputting information, measured values, data or data sets by means of an interface.

The data input unit preferably has interface elements, for example, amplifiers, A/D converters, components for overvoltage protection (ESD protection), logic elements and additional electronic components for the wired or wireless reception of the data and signals, as well as adaptation elements such as code or protocol conversion elements for adaptation of the signals and data for the further processing in the control unit.

The data output unit is configured for generating and providing the stimulation signal or an output signal.

The stimulation signal is preferably configured as a signal which is configured for controlling the stimulation device directly by means of a data line or via interfaces, bus systems (RS232, CAN bus, I²C bus, SPI, USB, SCSI, IEEE488), network systems (Ethernet, LAN, WLAN). The output signal is preferably configured as a video signal (e.g., Video Out, Component Video, S-Video, HDMI, VGA, DVI, RGB) to make possible graphic, numeric or pictorial representations on a display unit connected in a wireless or wired manner (WLAN, Bluetooth, WiFi) to the output unit or on the output unit itself.

According to another aspect of the present invention, the objects set are accomplished by a system with a data input unit, with a control unit, with a data output unit, with a stimulation device and with a measuring device. The measuring device comprises at least one pressure sensor and at least one flow rate sensor. The measuring device is configured to detect measured values of the flow rate sensor and to detect measured values of the pressure sensor. The measuring device is configured to provide information which indicates a ventilation pressure and/or a flow rate to the control unit.

The data input unit is configured to provide at least one piece of information to the control unit.

The control unit is configured to generate a stimulation signal on the basis of the at least one piece of information.

The data output unit is configured to provide the stimulation signal to the stimulation device.

The stimulation device is configured to carry out the stimulation by means of the stimulation signal on the basis of and taking into consideration the at least one piece of information.

In embodiments, the at least one piece of information may include measured values or data from other devices, as has been described before within the framework of the description of the present invention regarding aspects of the process according to the present invention. The embodiments related to these other devices are therefore explained only briefly at this point. Corresponding aspects, which are described in relation to the process according to the present invention, shall nevertheless also apply to the system according to the present invention.

These include, for example, devices for an imaging analysis or diagnosis, devices for blood analysis or diagnosis, devices for blood gas analysis or diagnosis, devices for determining an oxygen saturation or oxygen concentration in the blood, devices for determining an oxygen concentration in breathing gases, devices for a carbon dioxide saturation or carbon dioxide concentration in the blood, and devices for an invasive or non-invasive blood pressure measurement. Devices for an imaging analysis or diagnosis are, for example, devices for an electrical impedance tomography (EIT), devices for magnetic resonance imaging (MRI), devices for computer tomography (CT), and devices for ultrasound imaging. These other devices can provide the at least one piece of information, for example, in a network linking system (PAN, Ethernet, LAN, WLAN, Bluetooth) or as data input via the data input unit. In embodiments, the at least one piece of information may comprise data on types of ventilation modes, a manner of feeding breathing gases to the living being or patient, ventilation parameters, ventilation settings, settings on the ventilator, alarm settings, alarm limit settings on the ventilator with alarm threshold values, which can be provided, for example, in a network linking system (PAN, Ethernet, LAN, WLAN, Bluetooth) via the data input unit. The at least one piece of information may also comprise in embodiments reports, warnings, alarm generations from the ventilator, which may be provided, for example, in a network linking system (PAN, Ethernet, LAN, WLAN, Bluetooth) via the data input unit.

The at least one piece of information may comprise in embodiments data on individual information in relation to properties of the living being, such as general and physical constitution, age, height, body weight, sex, clinical picture, course of the disease, past medical history, diagnoses, and findings, which are provided, for example, in a network linking system (PAN, Ethernet, LAN, WLAN, Bluetooth) via the data input unit.

The at least one piece of information may comprise in embodiments a type of a ventilation mode currently being used during the ventilation of the living being, which can be provided, for example, in a network linking system (PAN, Ethernet, LAN, WLAN, Bluetooth) or by means of a manual data input via the data input unit.

In a preferred embodiment, the control unit is configured to bring about a control, selection, activation or deactivation of a first mode of operation of a follow mode as well as of a second mode of operation of the lead mode at the stimulation device by means of the stimulation signal. Depending on the conditions and on the basis of the at least one piece of information, especially information concerning the ventilation modes employed (volume-controlled/pressure-controlled ventilation modes), the control unit can in this manner shift the stimulation device into the follow mode or lead mode. Concerning the differences and aspects of the follow mode and lead mode, reference shall be made to the explanations given for the process according to the present invention within the framework of the description of the present invention.

Corresponding aspects of the follow mode and lead mode, which are described in connection with the process according to the present invention, shall nevertheless apply to the system according to the present invention as well.

In a preferred embodiment, the control unit is configured to include measured values of a flow rate or a flow rate sensor for controlling the carrying out of the stimulation if the at least one piece of information indicates that the ventilator is operating in an operating state of a volume-controlled ventilation.

In a preferred embodiment, the control unit is configured to include measured values of a ventilation pressure or a pressure sensor for controlling the carrying out of the stimulation if the at least one piece of information indicates that the ventilator is operating in an operating state of a pressure-controlled ventilation.

In a preferred embodiment, the control unit is configured to include measured values of a ventilation pressure or a flow rate sensor for controlling the carrying out of the stimulation if the at least one piece of information indicates that the ventilator operates in an operating state of a pressure-controlled ventilation.

A flow rate sensor may be used to determine a situation with the lungs filled approximately with fresh breathing gases during a progression of the inhalation phase. Such a situation can be detected by means of the cycling off criterion. A situation is detected here in which there is a drop of the flow rate currently detected by means of the flow rate sensor to a value of, e.g., about 15% to 25% of a value of the maximum inspiratory flow rate and the feed of additional quantities of breathing gases has ended. The lungs are filled nearly completely with the desired volume of breathing gases in such a situation.

Other embodiments show how a switching between modes of operation of the stimulation device or an adaptation during the carrying out of the stimulation, a transition from a stimulation in the follow mode to a stimulation in the lead mode can be configured by the control unit. For example, the control unit can thus initiate a switching between the first mode of operation of the follow mode and the second mode of operation of the lead mode at the stimulation device on the basis of the at least one piece of information.

The control unit can thus initiate an adaptation of the carrying out of the stimulation on the basis of a change in at the at east one information.

The control unit can thus initiate an iterative transition with a stimulation from the follow mode to a stimulation in the lead mode.

The control unit can thus initiate at the ventilator a maneuver for a transition from a stimulation to a stimulation into the lead mode.

Possibilities of combinations of the follow mode as well as of combinations of the lead mode with different ventilation modes will be presented and explained below. The follow mode can advantageously be combined with mandatory ventilation. Mandatory ventilation modes with predefined, unambiguous and structured ventilation patterns can be readily combined with the follow mode, because the stimulation ventilation can follow the ventilation pattern directly and reproducibly. Ventilation modes with support of spontaneous breathing of the patient cannot fully develop the advantages of the spontaneous breathing support in connection with the follow mode, whereas the lead mode can be readily combined in an advantageous manner with many ventilation modes, which offer a spontaneous breathing support.

The application of especially pressure-controlled or pressure-regulated ventilation modes with spontaneous breathing support is especially advantageous in this connection. Whenever no sufficient volume can be applied into the lungs by pressure-supported stimulation in the course of the ventilation, the missing volume can be complemented with a mandatory ventilation stroke. For example, ventilation modes such as PC-PSV (Pressure Control Pressure Support Ventilation) or SIMV (Synchronized Intermittent Mandatory Ventilation) may be mentioned in this connection. In the lead mode, the ventilator follows the stimulated contraction of the diaphragm, which acts as a spontaneous breathing.

Based on suitable configurations on the ventilator, the effect of the stimulation can be set such that the ventilator determines the ventilation to the lowest possible extent only, for example, with mandatory strokes on the basis of the setting of the backup frequency, and the spontaneous breathing brought about by stimulation provides a sufficient supply of breathing gases. The lead mode offers now, in addition, the advantage that a so-called “natural negative-pressure breathing” takes place by initiation of the inhalation at the patient before the beginning of the inhalation phase initiated by the ventilator, whereby the ventilator must build up a lower additional pressure difference for opening the lungs and for reaching the desired tidal volume and high values of the ventilation pressure are thus advantageously avoided, in principle., by the stimulation.

In case of application of the stimulation in the follow mode with pressure-controlled as well as volume-controlled ventilation modes, the patient carries out a part of the work of breathing due to the spontaneous breathing activated by means of stimulation. The flow rate and the total volume fed to the patient remain the same in volume-controlled ventilation modes compared to an application without stimulations, a reduction of the airway pressure (PAW) is achieved by means of the stimulations, and there are no pressure increases or excessive pressure increases in the lungs due to the stimulation in volume-controlled ventilation modes. This represents an advantage of a combination of volume-controlled ventilation and the carrying out of a stimulation of the nervous system, especially of the phrenic nerve, with activation of the respiratory muscle or auxiliary respiratory muscles.

When using a combination of pressure-controlled ventilation and carrying out of a stimulation of the nervous system, especially of the phrenic nerve, a limitation of the volume fed to the patient during the phase of inhalation can be brought about by means of a suitable setting of a volume upper limit in order to avoid an increase in volume or an excessive increase in volume, which is possible due to the increase in the driving pressure brought about by the stimulation.

At the end of the description of the embodiments and the explanations given for the follow mode and lead mode, it shall be described as an example in a brief review for some ventilation modes listed in Tables 1 through 5 for which of these ventilation modes especially practical and readily applicable combinations with carrying out of a stimulation of the nervous system, especially of the phrenic nerve, can be expected. Advantageous aspects can be expected in the application for the follow mode in combination with the following ventilation modes: VC-SIMV, VC-CMV, PC-CMV, PC-BiPAP, PC-SIMV, and PC-APRV. Advantageous aspects can be expected in the application for the lead mode in combination with the following ventilation modes: VC-SIMV, VC-AC, VC-MMV, PC-CMV, PC-SIMV, PC-BIPAP, PC-AC, PC-PSV, SPN-CPAP, and SPN-PPS.

Other embodiments can show how the control unit in the system can also influence the ventilation modes. Thus, the control unit may be configured to generate a control signal for controlling a maneuver at the ventilator. Furthermore, the control unit may be configured such as to generate a control signal for an activation or deactivation of a ventilation modes at the ventilator and to provide it to the ventilator by means of the data output unit. Furthermore, the control unit may be configured to generate at the ventilator a control signal for activation of a triggering of an automobile or for triggering for a supply of breathing gases in living beings and to provide it by means of the data output unit. The combination of ventilator and stimulation device in the system can influence in this manner both the stimulation and the manner of ventilation and of the supply of a gas, e.g., the selection of or the switching between different ventilation modes. Both a switching between follow mode and lead mode of the stimulation device and a selection of a pressure-controlled or volume-controlled ventilation mode is thus made possible for the operation of the ventilator.

Other embodiments can show how the control unit can be configured in the system or device.

Other embodiments can show how the measuring device can be configured in the system or device.

Other embodiments can show how the stimulation device may be configured for arrangement at the body of the living being with coupling of the stimulation into or onto the nervous system of the living being.

The control unit may be configured as an element of the ventilator, as an element of the stimulation device as well as a central control unit. The measuring device may be configured as an element of the ventilator, as an element of the stimulation device, as a central measuring device, as a combination of the at least one pressure sensor with the at least one flow rate sensor. In addition, the measuring device may be configured as a combination of the control unit with the at least one pressure sensor and of the at least one flow rate sensor. The stimulation device may be configured as an electrode array or as a coil array. An electrode array is configured here such that stimulation signals can be coupled into the body of the living being via electrodes, electrical signals or an electrical field. A coil array is configured in this case such that stimulation signals can be coupled into the stimulation via a magnetic field.

The described embodiments of the process according to the present invention, of the device according to the present invention as well as of the system according to the present invention represent each in itself as well as in combination with one another special embodiments of the device according to the present invention. Advantages arising through the combination or combinations of a plurality of embodiments and other embodiments are nevertheless covered by the inventive idea, even though not all the possibilities of combinations of embodiments are explained for this in detail.

The present invention will be explained in more detail now by means of the following figures and the corresponding descriptions of the figures without limitations of the general inventive idea.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a system with stimulation device and ventilator;

FIG. 2 is a schematic view showing a system with stimulation device, measuring device, control unit, data output unit, data input unit and ventilator;

FIG. 3 is a view with graphs showing signal/time courses (curves) in the course of the ventilation;

FIG. 4 is a view with graphs showing signal/time courses (curves) in the course of the ventilation;

FIG. 5 is a view with graphs showing signal/time courses (curves) in the course of the ventilation; and

FIG. 6 is a view with graphs showing signal/time courses (curves) in the course of the ventilation.

### DESCRIPTION OF PREFERRED EMBODIMENTS

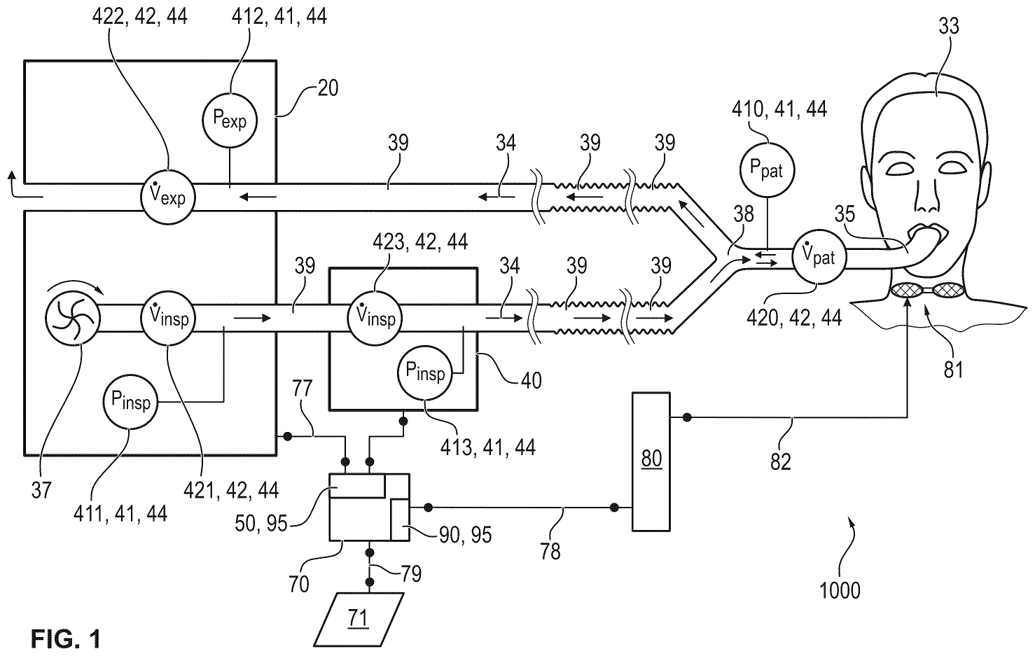

Referring to the drawings, FIG. 1 shows a schematic view of a system 1000 with a ventilator 20 and with a stimulation device 80. The system is a gas-carrying system with a ventilation drive 37 with gas-carrying elements 39 and with a patient-side connection element 38 for feeding breathing gases to a patient 33 or living being. Flow arrows indicate how quantities of gas flow in the gas-carrying system. The gas-carrying elements 39 have an inspiratory path with an inspiratory ventilation tube and an expiratory path with an expiratory ventilation tube. The inspiratory path and the expiratory path are connected to one another at the patient-side connection element 38 for connection to the bronchial tract of the patient 33 or living being. The breathing gases are fed to and removed from the patient 33 or living being from the patient-side connection element (Y-piece) 38 via a patient-side gas feed element 35, which may be configured as an endotracheal tube, nasal cannula, face mask, or tracheostomy.

An arrangement 40 of a sensor mechanism for pressure and flow measurement in an embodiment as an inspiratory pressure sensor 411, as an expiratory pressure sensor 412, as an inspiratory flow rate sensor 421, as an expiratory flow sensor 422 for detecting measured values 44, 41, 42, which is incorporated in, arranged at or associated with the ventilator 20 and/or with the stimulation device 80, is shown. The sensor mechanism arranged in the ventilator 20 for pressure and flow measurement is used to control the ventilation with the feed of breathing gases to a patient or living being 33. The pressure and flow measurement associated in or at or with the stimulation device 80 is used to control the stimulation of the living being by means of the patient-side stimulation arrangement 81. As an alternative to the arrangement of the sensor mechanism associated in, at or with the ventilator 20, a measuring device 40 may also be arranged in the system 1000 with an incorporated, attached or associated arrangement of the sensor mechanism in a configuration with an inspiratory pressure sensor 413 and of an inspiratory flow sensor 423.

As an alternative or in addition to the arrangement of the sensor mechanism incorporated in, at or associated with the ventilator 20, a patient-side flow rate sensor 423 may also be arranged close to the patient or living being 33 in the system 1000 at the patient-side connection element (Y-piece) 38.

As an alternative or in addition to the arrangement of the sensor mechanism incorporated in, at or associated with the ventilator 20, a patient-side pressure sensor 410 may also be arranged close to the patient or living being 33 in the system 1000 at the patient-side connection element (Y-piece) 38.

Furthermore, a sensor for detecting an oxygen saturation (SpO₂) 48 in the blood, a sensor for detecting an oxygen concentration (FiO₂) 47 in the exhaled gas or a sensor for detecting a carbon dioxide concentration (etCO₂) 49 in the exhaled gas may be arranged.

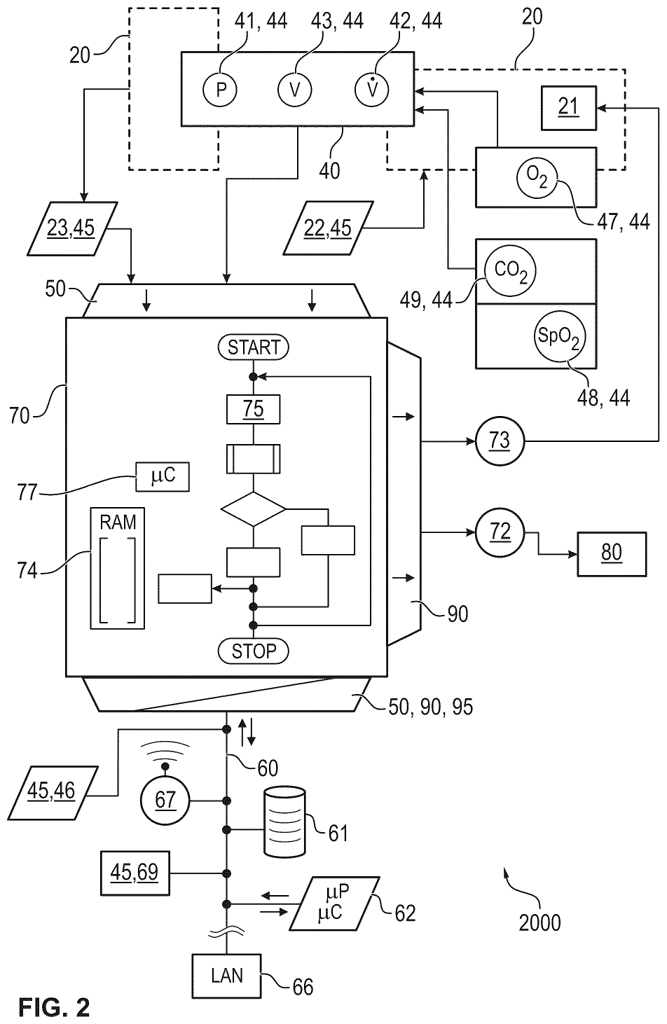

FIG. 2 shows a system 2000 with stimulation device 30, measuring device 40, control unit 70, data output unit 90, data input unit 50, stimulation device 80 and ventilator 20. Identical elements in FIGS. 1 and 2 are designated by the same reference numbers in FIGS. 1 and 2. The ventilator 20 has elements for dispensing gas (valves, breathing gas drive) 21, elements for carrying out settings 22 for operating the ventilator 20, such as a user interface (keyboard, switch, button, touchscreen) for entering information 45. The ventilator 20 has, furthermore, elements for outputting 23 alarm reports and warnings (user interface, sound-generating elements, screen). Alarm messages or warnings 23 may also be provided to the control unit 70 by means of data connection and input unit 50. The control unit may be connected to the stimulation device via a data line 78. The control unit 70 may optionally be connected to the ventilator 20 via a data line 77. The control unit 70 may be configured by means of an additional data line 79 for detection of information provided by manual inputs 71 by a user. A great variety of information can thus be provided for the stimulation device 80. This information may be used by the control unit 70 to carry out the control of the stimulation device 80 on a case-by-case basis on the basis of the pressure measurement 413 or on the basis of the flow measurement 423 with selection of the corresponding sensor mechanism 40, 44 suitable for this.

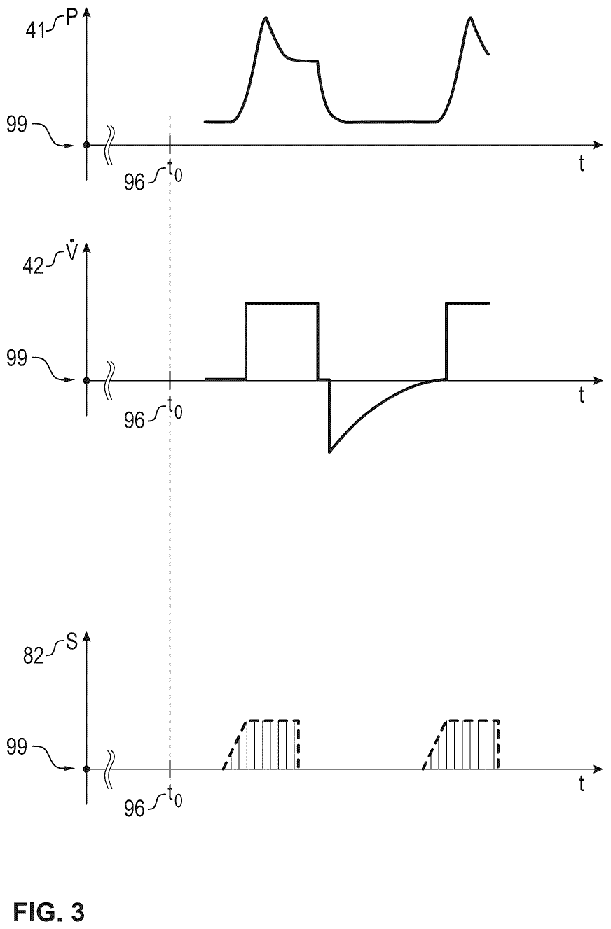

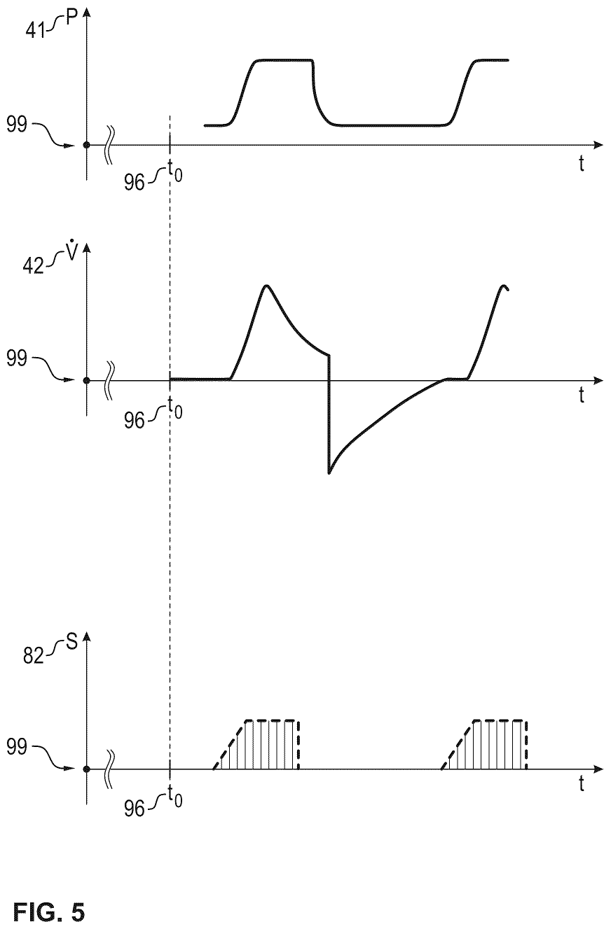

If, for example, a piece of information is provided by means of manual input 71 or by the ventilator 20 that the ventilator 20 is operating in an operating state of a volume-controlled ventilation of the patient 33 and a volume-controlled ventilation is being carried out, measured values of the flow measurement 423 are included during the carrying out of the stimulation by the stimulation device 80. If, for example, a piece of information is provided by means of manual input 71 or by the ventilator 20 that the ventilator 20 is operating in an operating state of a pressure-controlled ventilation of the patient 33 or a pressure-controlled ventilation is being carried out, measured values of the pressure measurement 413 are included during the carrying out of the stimulation by the stimulation device 80. The control of the stimulation can thus take place with inclusion of measured values 44. During a control of the stimulation in the follow mode, a characteristic change in the course of the pressure measured value 413 at the end of the inhalation phase prior to the beginning of the expiration over the time course of the ventilation is used to deactivate the stimulation during a pressure-controlled ventilation, as is shown in FIG. 5. During the control of the stimulation in the follow mode, a characteristic change over the course of the ventilation in the volume increase or a drop in the measured value of the inspiratory flow rate 423 by the end of the inhalation phase prior to the beginning of the expiration is used to deactivate the stimulation during a control of the stimulation in the follow mode in case of a volume-controlled ventilation, as is shown in FIG. 3. The deactivation or a stop of the stimulation thus takes place in the follow mode reliably and with certainty as soon as the set and desired volume of breathing gases is fed to the patient 33. Such a deactivation may take place on the basis of the measuring device 40, 44 associated by the sensor mechanism 413, 423 with the stimulation device 80, on the one hand, and, on the other hand, information 411, 421 provided, which indicate pressure and/or flow rates and are provided by the ventilator 20, bring about the deactivation or stop of the stimulation in the follow mode reliably and with certainty in order to ensure that stimulations do not take place nor are continued after set quantities of breathing gases have been reached. Risks due to stimulations at unsuitable times can thus be avoided or reduced with certainty in the follow mode.

A sensor mechanism 423, 413, which is directly associated with the stimulation device 80 or is configured as a part of the stimulation device 80, offers the advantage that no data connection to the ventilator 20 is necessary. Such a configuration of the stimulation device 80 can also be operated in combination with ventilators, which do not provide any data interface for information such as measured pressure values or measured flow rate values and they usually cannot be easily upgraded at the user's premises in terms of the data supply. The sensor mechanism 423, 413 in the measuring device 40 with the measuring device as a module of the stimulation device makes it possible that the stimulation can be synchronized with the time window of the inhalation phase independently from a data supply by a ventilator.

A sensor mechanism, 421, 411, 41, 42, which is configured as a part of the ventilator 20, and whose information is provided to the stimulation device 80 by means of data interfaces, offers the advantage of configuring a system 2000 as an integrative or modular solution, comprising a ventilator 20 and a stimulation device 80, and of making it possible to eliminate the need for redundant elements in terms of a sensor mechanism, as well as measured value and signal processing. Another advantage of such a system configuration 2000 arises from the fact that the data communication can be organized internally and the sensor signals are available in this manner—without processing or conversion for being made available in a data network—in real time, without essential delays and synchronously in time for the control for carrying out the ventilation and for the control of the stimulation at the same time.

The control unit 70 may be arranged centrally in the system 2000 or be configured and arranged in the system 2000 in a modularly distributed manner, both as a module or partial module of the stimulation device 80, the measuring device 40, the control unit 70, the data output unit 90, the data input unit 50, the ventilator 20 as well as a central control unit. Divisions into master/slave configurations are possible in the system 2000. The system 2000 may have as interfaces 60 a network (LAN, WLAN, Ethernet) or a bus system (RS232, CAN-bus, I²C-bus, SPI, USB, SCSI, IEEE488), via which the components 20, 40, 50, 60, 70, 80, 90 can be connected for a unidirectional or bidirectional data exchange in the system 2000. The control unit 70 can now initiate, coordinate or control the carrying out and an adaptation of the carrying out of the stimulation by means of the stimulation signal 72 at the stimulation device 80.

The control unit 70 can in this case initiate, coordinate or control the carrying out of a ventilation by means of the control signal 73 at the ventilator 20. The control unit has for this purpose suitable elements, such as a processor (P) 77 or a microprocessor (P) 77 or even a microcontroller (C) 77, a main memory (RAM), a program memory with program code 75. An initiation makes it possible for the control unit 70 to trigger or activate elements in or at the stimulation device 80 or at the ventilator 20. A coordination makes it possible for the control unit 70 in the system 2000 to coordinate measured value acquisition, inclusion of sensors or actuators, data processing and activation of the setting or switching elements for the carrying out of the stimulation as well as the adaptation of the carrying out of the stimulation. Controlling makes it possible for the control unit 70 to control (open loop control), for example, to adjust or set, setting or switching elements of the system 2000, of the stimulation device (80) or of the gas-dispensing elements 21 of the ventilator 20. Controlling makes it possible for the control unit 70 to regulate or control (closed loop control) by means of adjustment or setting of setting or switching elements in the system 2000, in the stimulation device 80 or of the gas-dispensing elements 21 in the ventilator 20 in a closed control circuit and a configuration of a regulation or of a controller of a certain type (e.g., as a PID controller with amplification K_{P}, adjustment time T_{N}, rate time T_{V}).

Additional information, for example, information or data on gas concentrations in inhaled gases, exhaled gases or blood, information or data on gas concentrations in the blood, can be provided to the control; unit 70 via interfaces 60 and/or networks (LAN 66), (WLAN) 67. These include, for example, among other things, carbon dioxide concentrations (etCO₂) 49 (FIG. 1) in the exhaled gas, oxygen saturation (SpO₂) in the blood 48 (FIG. 1) or oxygen concentration (FiO₂) 47 (FIG. 1) in the inhaled gas.

Information in respect to a situation of the ventilation being currently carried out may also be provided as additional information. Such additional information may also be provided in the system 2000 via interfaces 60, for example, by devices for an imaging analysis or diagnosis, such as devices for an electrical impedance tomography or EIT systems, devices for magnetic resonance imaging (MRI), devices for computed tomography (CT), and devices for ultrasound imaging.

Information in respect to an individual situation of a patient 33 (FIG. 1) can also be provided as additional information 45 via the network 60, 66, 67 from a hospital management system or patient data management system (database) 69 to the control unit 70.

Manual inputs 46 may comprise information 45 on the individual situation of a patient 33 (FIG. 1) and on an operating state and the ventilation mode of the ventilator 20. The network 66 may, in addition, also comprise additional components, such as storage media (hard drives) 61, computers (servers) 62.

It may be advantageous for an implementation of an adaptation of the carrying out of a stimulation during the ventilation of a living being 33 (FIG. 2) if the ventilator 20, the stimulation device 80, the measuring device 40, the control unit 70, the data output unit 90, the data input unit 50 and possibly also additional optional components, such as a sensor for detecting an oxygen saturation (SpO₂) in the blood or carbon dioxide concentration (etCO₂) in the exhaled gas, which sensor is not shown in this figure, and the system 2000 thus form a common system in a functional manner.

The data output unit 90 and the data input unit 50 may also be configured together as a data input and output unit 95.

FIGS. 3 through 6 show schematic signal/time curves (time course) 99 with time axes 99 (x axis, abscissas) synchronized with one another at a time to 96 in the course of the ventilation, with time courses of the ventilation pressure 41 (Pᵢₙₛₚ, Pₑₓₚ, P_{AW}, PEEP), flow rates 42 (Flowᵢₙₛₚ, Flowₑₓₚ) and of activation signals S 82 for a muscle stimulation of breathing efforts of a living being or patient 33 (FIG. 1). Identical elements in FIGS. 1, 2, 3, 4, 5 and 6 are designated by the same reference numbers in FIGS. 1, 2, 3, 4, 5 and 6. FIGS. 3 through 6 are shown in a joint description of the figures, emphasizing the differences, in more detail. The activation signals S 82 for the stimulation are shown here schematically in FIGS. 3-6 as a sequence of pulses. The pulses for coupling to the living being 33 (FIG. 1) are configured in typical embodiments as sine pulses, especially as magnetic field pulses with a pulse duration in the range of 150 μsec to 300 μsec.

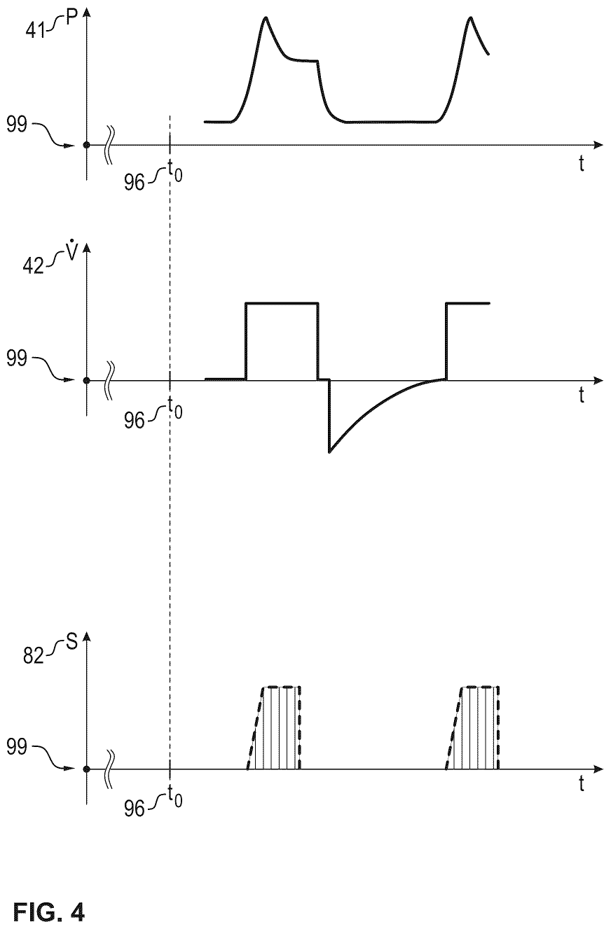

FIG. 3 and FIG. 4 show typical and exemplary time courses 99 of a carrying out of a volume-controlled ventilation.

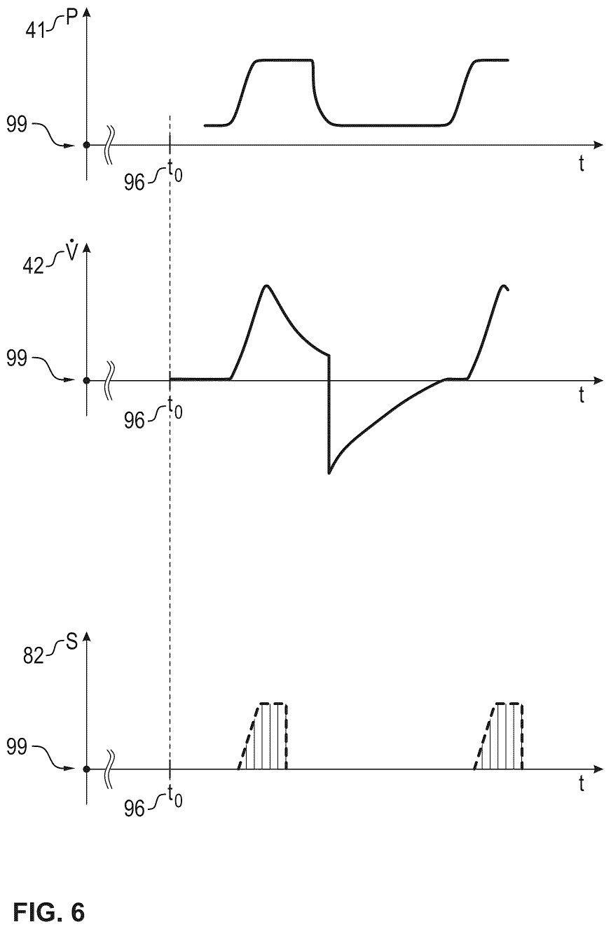

FIG. 5 and FIG. 6 show typical and exemplary time courses 99 of a carrying out of a pressure-controlled ventilation.

FIG. 3 and FIG. 5 show typical and exemplary time courses 99 of a carrying out of a stimulation in a follow mode.

When the stimulation is carried out in the follow mode, the activation of the stimulation signal S takes place after the onset of an inhalation by the living being 33 (FIG. 1), after an inhalation trigger or after the initiation of a phase of inhalation. The initiation of the inhalation phase (inspiration phase) can be brought about by the ventilator 20 (FIG. 1) or by an inhalation effort of a patient 33 (FIG. 1). FIG. 3 shows the stimulation S 82 during a carrying out of a volume-controlled ventilation. FIG. 5 shows the stimulation S 82 during a carrying out of a pressure-controlled ventilation.

FIG. 4 and FIG. 6 show typical and exemplary time courses 99 of a carrying out of a stimulation in a lead mode.

When the stimulation is carried out in the lead mode, a direct activation of a muscle breathing effort of a patient 33 (FIG. 1) and hence indirectly also an activation of the ventilator 20 (FIG. 1) take place for providing breathing gases according to the particular ventilation mode carried out by the ventilator on the basis of the triggerings elicited by the breathing effort at the ventilator. Both pressure triggerings, i.e., triggerings based on changes in the inspiratory ventilation pressure, as well as flow triggerings, i.e., changes in the inspiratory flow rates based on triggerings, can be employed at the ventilator 20 (FIG. 1) as possibilities of triggering.

FIG. 4 shows the stimulation S 82 during a carrying out of a volume-controlled ventilation.

FIG. 6 shows the stimulation S 82 during a carrying out of a pressure-controlled ventilation.

The stimulations S 82 with an application in the follow mode (FIGS. 3, 5) differ from the stimulation with an application in the lead mode (FIGS. 4, 6) in that a longer duration is available for the stimulation in the lead mode, during which a number of excitation pulses are applied to the stimulation device at the body of the patient 33 (FIG. 1) than in the follow mode (FIGS. 3, 5). A time interval is available as an available duration for the stimulation, which must be clearly and reliably terminated prior to a possible onset of the exhalation or of an exhalation effort must have ended in order for a stimulation to be avoided in any case during an exhalation by the patient 33 (FIG. 1). As a result, a longer duration of the stimulation can be selected in the lead mode (FIGS. 4, 6), for example, even in combination with a ramp-like rise of the activation signal S 82. In addition, due to the availability of the longer duration for achieving an effect of the stimulation, the amplitude of the activation signal S 82 can be selected to be smaller in the lead mode (FIGS. 4, 6) than in the follow mode (FIGS. 3, 5). The beginning of the activation signal S 82 can ideally be selected in the lead mode (FIGS. 4, 6) at a time in the course of the ventilation, at which the exhalation of the preceding ventilation cycle has largely ended. This time of termination of the exhalation by the patient 33 can be detected by measurement, for example, by means of a flow rate sensor, which is configured for detecting the direction and the quantity of exhaled gases.

While specific embodiments of the invention have been shown and described in detail to illustrate the application of the principles of the invention, it will be understood that the invention may be embodied otherwise without departing from such principles.

### LIST OF REFERENCE NUMBERS

20 Ventilator
21 Gas-dispensing elements (valves, breathing gas drive)
22 Elements for settings for the operation
23 Elements for alarm reports, warnings
33 Living being, patient
34 Flow arrows
Patient-side gas feed element
(endotracheal tube, nasal cannula, face mask, tracheostomy)
37 Ventilation drive, radial compressor (blower)
38 Patient-side connection element (Y-piece)
39 Gas-carrying elements, ventilation tubes
40 Measuring device
41 Measured pressure values (airway pressure, ventilation pressure)
411 Inspiratory pressure sensor in the ventilator
412 Expiratory pressure sensor in the ventilator
413 Inspiratory pressure sensor in the measuring device
410 Patient-side pressure sensor
42 Measured flow values (breathing gases)
421 Inspiratory flow rate sensor in the ventilator
422 Expiratory flow rate sensor in the ventilator
423 Inspiratory flow rate sensor in the measuring device
420 Patient-side flow rate sensor
43 Measured volume values (breathing gas)
44 Measured values, measured data
At least one piece of information, information
46 Input, manual data input
47 Measured oxygen values (inhaled gas)
48 Measured oxygen saturation values (blood)
49 Measured carbon dioxide values (exhaled gas)
50 Data input unit
60 Interfaces, bus system, data bus
61 Storage media (hard drives)
62 Computers (servers)
66 LAN, network, Ethernet
67 WLAN, wireless network
69 Hospital management system
Patient data management system, (database)
70 Control unit
71 Data input
72 Stimulation signal
73 Control signal
74 Memory RAM
75 Program code, run
77, 78, 79 Data lines
80 Stimulation device
81 Patient-side stimulation device (coil array, electrode array)
82 Activation signals S for stimulation
90 Data output unit
95 Data input/output unit
96 Time to
99 Signal courses
1000 Device
2000 System

1. A process for generating and providing a stimulation signal for carrying out a stimulation influencing the nervous system of a living being, based on information, which indicates a state of a breathing activity or a state of a ventilation and/or an operating state of a ventilator, the process comprising the steps of:
carrying out of the stimulation by means of a stimulation signal on the basis of and taking into account the information;
controlling the stimulation with, a selection, activation or deactivation of modes of operation at a stimulation device by means of the stimulation signal;
wherein a stimulation and influencing of the phrenic nerve is carried out with the carrying out of the stimulation with the influencing of the nervous system of the living being by means of the stimulation signal;
wherein a control and synchronization of the stimulation and influencing of the phrenic nerve are carried out with a breathing activity of the living being or with a triggering of a breathing activity or ventilation of the living being on the basis of the information;
wherein the information indicates a type of a ventilation mode, wherein for controlling the synchronization of the stimulation,
if the information indicates that the ventilation is carried out in a volume-controlled ventilation mode, the information also indicates a flow rate or a volume, and
if the information indicates that the ventilation is carried out in a pressure-controlled ventilation mode, the information also indicates a ventilation pressure and/or also indicates a flow rate or a volume.

2. A process in accordance with claim 1,
wherein the information comprises measured values or data, which are provided by one or more of:
a device configured for an imaging analysis or diagnosis,
a device configured for a blood analysis, blood gas analysis or diagnosis,
a device configured for a determination of an oxygen saturation or of an oxygen concentration in the blood,
a device configured for a determination of an oxygen concentration in breathing gases,
a device configured for a determination of a carbon dioxide concentration in breathing gases,
a device configured for a determination of a carbon dioxide saturation or of a carbon dioxide concentration in the blood, and
a device configured for an invasive or non-invasive blood pressure measurement; wherein the at least piece of information comprises individual information in relation to properties of a living being or of a patient, such as age, body weight, height, sex, clinical picture, and/or
wherein the information comprises information from a group of information on settings, measured values or data concerning at least one of the type of a ventilation mode, such as volume-controlled or pressure-controlled ventilation modes,
types of ventilation modes,
manner of feeding breathing gases,
ventilation settings or parameters of the ventilator, such as ventilation frequency (RR), tidal volume (VT), minute volume (MV), inhalation to exhalation ratio (I:E ratio), breath volume, airway pressure, inspiratory ventilation pressure, expiratory ventilation pressure, positive end-expiratory pressure (PEEP),
the time of a beginning or of an end of the rising flank of an inspiratory ventilation pressure,
the time of a beginning or of an end of the plateau phase of the inspiratory ventilation pressure,
inspiratory oxygen concentration (FiO₂),
expiratory carbon dioxide concentration (etCO₂),
setting limits, alarm settings for alarm generations by the ventilator with upper limits and lower limits
of an expiratory minute volume,
of an airway pressure,
of an inspiratory O₂ concentration,
of an end-expiratory CO₂ concentration
of a volume monitoring,
upper limit of a tachypnea monitoring,
time range monitoring of an apnea alarm time, and
reports, warnings or alarm generations in case of exceeding/undershooting upper/lower limits and predefined time ranges.

3. A process in accordance with claim 1, wherein the control of the stimulation or a selection, activation or deactivation of a first mode of operation of a follow mode, and/or of a second mode of operation of a lead mode is carried out by means of the stimulation signal at the stimulation device.

4. A process in accordance with claim 3,
wherein, if the information indicates that the ventilation of the living being is carried out in an operating state with a pressure-controlled ventilation mode, the first mode of operation can be activated by the stimulation signal with a follow mode at the stimulation device, and
wherein, if the information indicates that the ventilation of the living being is carried out with a pressure-controlled ventilation mode, the second mode of operation can be activated by the stimulation signal with a lead mode at the stimulation device.

5. A process in accordance with claim 4, wherein if the ventilation is carried out in a pressure-controlled ventilation mode in combination with a stimulation of the nervous system for triggering a breathing effort of the living being, signals of a flow rate sensor and/or signals of a pressure sensor are used as inhalation triggers in the first mode of operation in the follow mode or in the second mode of operation in the lead mode.

6. A process in accordance with claim 5,
wherein, if the indication indicates that the ventilation of the living being is carried out in an operating state with a volume-controlled ventilation mode, the first mode of operation can be activated by the stimulation signal with a follow mode at the stimulation device, and
wherein, if the information indicates that the ventilation of the living being is carried out in an operating state with a volume-controlled ventilation mode, the second mode of operation can be activated by the stimulation signal with a lead mode at the stimulation device.

7. A process in accordance with claim 6, wherein if the ventilation is carried out in a volume-controlled ventilation mode in combination with a stimulation of the nervous system for triggering a breathing effort of the living being, signals of a flow rate sensor and/or signals of a pressure sensor are used as inhalation triggers for triggering a breathing effort of the living being in the first mode of operation in the follow mode or in the second mode of operation in the lead mode.

8. A process in accordance with claim 3,
wherein an adaptation of the carrying out of the stimulation is carried out in the course of the ventilation on the basis of changes in the information, and
wherein a switching from the first mode of operation to the second mode of operation, an iterative transition with a stimulation from the follow mode to a stimulation in the lead mode, and a transition from a stimulation from the follow mode to a stimulation in the lead mode are carried out by means of a maneuver as an adaptation of the carrying out of the stimulation.

9. A process according to claim 1, further comprising providing a computer program or computer program product with a program code on non-transitory computer readable storage medium for carrying out one or more of the processes steps when the program code is executed on a computer, on a processor or on a programmable hardware component.

10. A device, for generating and providing a stimulation signal for carrying out a stimulation influencing a nervous system of a living being, the device comprising:
a data input unit providing information which indicates a state of a breathing activity or a state of a ventilation and/or an operating state of a ventilator and that indicates a type of a ventilation mode, for controlling a synchronization of the stimulation, wherein if the information indicates that the ventilation is carried out in a volume-controlled ventilation mode, the information also indicates a flow rate or a volume, and if the information indicates that the ventilation is carried out in a pressure-controlled ventilation mode, the information also indicates a ventilation pressure and/or indicates a flow rate or a volume;
a control unit configured to generate the stimulation signal on the basis of the information, and to control stimulation with, a selection, activation or deactivation of modes of operation with the stimulation signal, wherein a stimulation and influencing of the phrenic nerve is carried out with the carrying out of the stimulation with the influencing of the nervous system of the living being by means of the stimulation signal and wherein a control and synchronization of the stimulation and influencing of the phrenic nerve are carried out with a breathing activity of the living being or with a triggering of a breathing activity or ventilation of the living being on the basis of the information;
a data output unit providing the generated stimulation signal; and
a stimulation device receiving the stimulation signal from the data output unit.

11. A device according to claim 10 in combination with a measuring device to form a system, the measuring device comprising at least one pressure sensor and at least one flow rate sensor,
wherein the measuring device is configured for detecting measured values of the flow rate sensor,
wherein the measuring device is configured for detecting measured values of the pressure sensor,
wherein the measuring device is configured for providing information which indicates a ventilation pressure and/or a flow rate,
wherein the data input unit is configured for providing information to the control unit,
wherein the control unit is configured for generating a stimulation signal on the basis of the information,
wherein the data output unit is configured for providing the stimulation signal to the stimulation device, and
wherein the stimulation device is configured for carrying out a stimulation by means of the stimulation signal on the basis of and taking into consideration the information for carrying out the stimulation by means of the stimulation signal.

12. A system in accordance with claim 11,
wherein the information comprises measured values or data
of a device configured for an imaging analysis or diagnosis,
of a device configured for a blood analysis, blood gas analysis or diagnosis,
of a device configured for determining an oxygen saturation or an oxygen concentration in the blood,
of a device configured for determining an oxygen concentration in breathing gases,
of a device configured for determining a carbon dioxide concentration in breathing gases,
of a device configured for determining a carbon dioxide saturation or a carbon dioxide concentration in the blood,
of a device configured for an invasive or non-invasive blood pressure measurement, or
wherein the information comprises measured values or data of a ventilator concerning
a type of a ventilation mode currently being carried out during the ventilation of the living being, types of ventilation modes, a manner of feeding breathing gases to the patient,
ventilation settings or parameters of the ventilator,
setting limits, alarm settings for alarm generations by the ventilator with upper limits and lower limits, and
reports, warnings, alarm generations by the ventilator, or
individual information in relation to properties of the living being, such as age, body weight, height, sex, clinical picture, and
wherein the control unit is configured to bring about by means of the stimulation signal a control, selection, activation or deactivation of a first mode of operation of a follow mode and/or of a second mode of operation of a lead mode at the stimulation device.

13. A system in accordance with claim 11,
wherein the control unit is configured to include measured values of a flow rate or a flow rate sensor for the control of the carrying out of the stimulation if the information indicates that the ventilator is operating in an operating state of a volume-controlled ventilation, and/or
wherein the control unit is configured to include measured values of a ventilation pressure or a pressure sensor for controlling the carrying out of the stimulation if the information indicates that the ventilator is operating in an operating state of a pressure-controlled ventilation, and/or
wherein the control unit is configured to include measured values of a flow rate or a flow rate sensor for controlling the carrying out of the stimulation if the information indicates that the ventilator is operating in an operating state of a pressure-controlled ventilation.

14. A system in accordance with claim 12,
wherein the control unit is configured:
to initiate a switching at the stimulation device between the first mode of operation of the follow mode and the second mode of operation of the lead mode at the stimulation device on the basis of the information,
to initiate an adaptation of the carrying out of the stimulation on the basis of changes in the information,
to initiate an iterative transition with a stimulation from the follow mode to a stimulation in the lead mode,
to initiate a maneuver at the ventilator for a transition from a stimulation from the follow mode to a stimulation into the lead mode;
and/or
wherein the control unit is configured to generate a control signal for controlling the maneuver at the ventilator and for activating or deactivating a ventilation mode on the basis of the information and to provide same the control signal to a ventilator by means of the data output unit.

15. A system in accordance with claim 11,
wherein the control unit is configured to generate a control signal for activating the triggering of a breath or a triggering for providing breathing gases to a living being and to provide the control signal by means of the data output unit for a ventilator,
wherein the control unit is at least one of configured
as an element of the ventilator,
as an element of the stimulation device, or
as a central control unit in the system,
wherein the measuring device may be configured
as an element of the ventilator,
as an element of the stimulation device,
as a combination of the at least one pressure sensor with the at least one flow rate sensor,
as a combination of the control unit with the at least one pressure sensor and with the at least one flow rate sensor,
as a central measuring device in the system, and
wherein the stimulation device is configured for being arranged at the body of the living being with coupling of the stimulation into or onto the nervous system of the living being with
an electrode array and/or
with a coil array.
